(19) United States
(12) Patent Application Publication
(10) Pub. No.: US 2021/0307317 A1
(43) Pub. Date: Oct. 7, 2021

(54) POLY-LYSINE DERIVATIVE STABILIZE SOLID-BASED COMPOSITIONS COMPRISING ONE OR MORE SALTS

(71) Applicant: BASF SE, Ludwigshafen am Rhein (DE)

(72) Inventors: Markus Kalt, Ludwigshafen (DE); Janine Rude, Ludwigshafen (DE); Helmut Witteler, Ludwigshafen (DE); Markus Meise, Ludwigshafen (DE); Frank Klippel, Ludwigshafen (DE); Tina Schroeder-Grimonpont, Ludwigshafen (DE); Hans-Peter Seelmann-Eggebert, Limburgerhof (DE)

(21) Appl. No.: 16/612,206

(22) PCT Filed: May 11, 2018
(86) PCT No.: PCT/EP2018/062197

§ 371 Date: Nov. 8, 2019

(30) Foreign Application Priority Data
May 12, 2017 (EP) 17170900.9
Sep. 25, 2017 (EP) 17192942.5
Dec. 21, 2017 (EP) 17209244.7

(51) Int. Cl. A01N 25/10; A01N 25/04; A01N 43/54; C08G 69/04; C08G 69/10; C08G 69/48; C05G 5/40

(52) U.S. Cl. CPC A01N 25/10; A01N 25/04; A01N 43/54; C05G 5/40; C08G 69/10; C08G 69/48; C08G 69/04

(57) ABSTRACT

Described herein is a polymeric stabilizing agent selected from poly-lysine derivatives obtained by process including heating an aqueous lysine solution to boiling, increasing a temperature of the aqueous lysine solution to a reaction temperature in the range of about 105° C. to about 180° C., and keeping the reaction temperature in the range of about 105° C. to about 180° C. until a melt viscosity of the reaction mixture is in the range of about 350 mPa*s to about 6,500 mPa*s, and (ii) an amine number in the range of about 100 mg KOH/g to about 500 mg KOH/g is achieved. The process also includes adding alkyl-carboxylic acid or alkenyl-carboxylic acid in amounts of 2.5 mol % to 10 mol %, and increasing or keeping the reaction temperature in the range of about 105° C. to about 180° C. until number of free alkyl-carboxylic acid or alkenyl-carboxylic acid is ≤9% by weight.

The present invention relates to storage-stable solid based compositions, a method of producing storage-stable solid based compositions by adding at least one derivative selected from poly-lysine functionalized with fatty acid(s) to such compositions, and to a method of stabilizing homogenous solid-based compositions comprising salts by adding at least one poly-lysine derivative selected from poly-lysine functionalized with fatty acid(s) to such compositions.

The present invention comprises combinations of preferred features with other preferred features.

There is a need for storage-stable solid-based compositions it the art of formulation. There is a special need for stabilizing agents for solid-based compositions comprising salts to avoid destabilization of said compositions due to effects such as agglomeration, flocculation, sedimentation, crystal growth and others.

A “stabilizing agent” in this context means a substance that establishes or at least maintains homogeneity in solid-based compositions.

“Storage-stable” in this context may mean that particles comprised in the solid-based composition don't grow, meaning that no significant increase in particle size of the dispersed solid compound (due to e.g. agglomeration) during storage over time. “Storage-stable” may mean that no gelling occurs, i.e. no increase in viscosity occurs during storage over time. “Storage-stable” may mean that once dispersed solid particles which have settled during storage over time are re-dispersible and don't clump. “Storage” usually means stock something over a certain time under certain storage conditions.

The object of the present invention was to provide a stabilizing agent that maintains distribution of solid phases within another phase even if salts are added. It was further an objective of the present invention, to provide a stabilizing agent which increases storage-stability of solid based compositions comprising salts.

The problem was solved by providing a polymeric stabilizing agent which is a non-crosslinked poly-lysine derivative selected from poly-lysine functionalized with fatty acid(s), wherein said poly-lysine derivative is obtained by a process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, the non-crosslinked poly-lysine derivative obtained by the inventive process is further processed by the following additional steps:
(g) vacuum applied is released and the temperature within the reaction mixture is reduced to about 150° C. to about 100° C.
(h) water is added to yield a poly-lysine derivative solution comprising about 60 parts polylysine derivative and about 40 parts water.

In one embodiment, the poly-lysine is modified prior to step (e) by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the non-crosslinked poly-lysine derivative obtained, is modified in step (g) by alkoxylation such as ethoxylation and/or reaction with monofunctional molecules such as amines, isocyanate, carboxylic acids, alcohols such as mPEG, thiols, esters, acid chlorides, anhydrides, and carbonates.

The invention further relates to a non-crosslinked poly-lysine oleate having a weight-average molecular weight in the range of about 20,000 g/mol to about 60,000 g/mol. In one embodiment, said poly-lysine oleate has a polydispersity index (PDI) of about 3.0 to about 10.0. In one embodiment, said poly-lysine oleate is water-soluble.

The invention further relates to a non-crosslinked poly-lysine laurate having a weight-average molecular weight in the range of about 20,000 g/mol to about 85,000 g/mol, or in the range of about 20,000 g/mol to about 60,000 g/mol. In one embodiment, said poly-lysine laurate has a polydispersity index (PDI) of about 3.0 to about 10.0. In one embodiment, said poly-lysine laurate is water-soluble.

In one aspect, the non-crosslinked poly-lysine functionalized with fatty acid(s) is used as a stabilizing agent for solid-based compositions. The non-crosslinked poly-lysine functionalized with fatty acid(s) may be used as a stabilizing agent, dispersing and wetting agent in solid-based compositions

The invention provides a storage-stable solid-based composition comprising
(1) a liquid phase comprising components A and B and one or more salts and optionally component C and optionally at least one additional solvent, wherein component A comprises at least one poly-lysine derivative obtained by the inventive process, wherein component B comprises a compound selected from the group of solvents in which component A is soluble, wherein component C is selected from at least one additional compound, wherein at least one additional solvent is immiscible with component B, and wherein component A and at least one salt are soluble in component B, and
(2) component D: comprising at least one solid compound, wherein component B is dispersed in the liquid phase.

In one embodiment, component D is solid at 20° C. and 101.3 kPa, and insoluble in component B.

In one embodiment, one or more salts are soluble in component B at 20° C. and 101.3 kPa until the saturation concentration is achieved.

In one embodiment, the liquid phase comprises one or more salts which are soluble in at least one additional solvent immiscible with component B at 20° C. and 101.3 kPa until the saturation concentration is achieved.

The invention relates to a method of producing a storage-stable solid-based composition comprising the steps of
I. providing a solution (1) by dissolving component A in component B, and optionally adjusting to pH of solution (1), and
II. providing a liquid (2) by dissolving one or more salts in at least one solvent, and optionally adjusting the pH of liquid (2), and
III. providing a solid-based composition (3) by dispersing component D in a dispersing medium comprising at least one dispersant in which component D is insoluble, and
IV. mixing at least the solution (1) and the solid-based composition (3) and optionally the liquid (2),

The method of producing a storage-stable solid-based composition of the invention may include the process of comminution, which takes place in step (III). The liquid (2) may comprises at least one salt dissolved in a solvent which is miscible with component B. The liquid (2) may comprise at least one solvent which is immiscible with component B.

The invention provides a method of stabilizing a solid-based composition comprising the steps of
(1) providing a solid-based composition comprising at least component D in a liquid dispersing medium, and optionally adjusting the pH of the solid based-composition, and
(2) providing a solution comprising component A dissolved in component B, and optionally adjusting the pH of the solution, and
(3) mixing (1) and (2),
   wherein component D is insoluble in the liquid dispersing medium.

In one embodiment, one or more salts are comprised in solubilized form in the dispersing medium. The dispersing medium and component B may be miscible with each other.

In one embodiment, the solid-based composition is mixed with a liquid phase comprising at least one solvent immiscible with component B, wherein the pH of this liquid phase is adjusted prior to mixing. The liquid phase comprising a solvent immiscible with component B may comprise at least one salt which is soluble in at least one solvent immiscible with component B. The invention provides a method of stabilizing a solid-based composition comprising the steps of
(1) providing a solid-based composition comprising at least component D in a liquid dispersing medium, wherein the liquid dispersing medium comprises component A dissolved in component B, and optionally adjusting the pH of the solid based-composition, and
(2) providing a solution comprising at least one salt, and optionally adjusting the pH of the solution, and
(3) mixing (1) and (2),
   wherein component D is insoluble in the liquid dispersing medium.

In one embodiment, the solid-based composition is mixed with a liquid phase comprising at least one solvent immiscible with component B, wherein the pH of this liquid phase is adjusted prior to mixing. The liquid phase comprising a solvent immiscible with component B may comprise at least one salt which is soluble in at least one solvent immiscible with component B.

The invention provides the use of at least one poly-lysine functionalized with fatty acid(s) in solid-based compositions comprising one or more salts to increase storage-stability of said composition when compared to compositions lacking the poly-lysine derivative of the invention stored under the same conditions.

Lysine, which is the monomer of poly-lysine, is characterized herein by specific positions of consecutive C-atoms starting from the carboxylic group using the Greek alphabet; alpha (α) C is located next to the lysine carboxylic group. It binds a primary amine group. α C is followed by beta (β), gamma (γ), delta (δ) and epsilon (ε) C, the latter binds a primary amine group. In other words, there are amine groups bound in alpha and epsilon position of the lysine molecule. Consequently, lysine molecules during polymerization may result in poly-lysine molecules with branching, when polymerization takes place in alpha and epsilon position.

The current invention relates to a polymeric stabilizing agent selected from poly-lysine derivatives, wherein the poly-lysine derivative obtained by a process comprising the steps of:
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture, wherein vacuum is applied either in step (a), (b) and/or (c) and water is removed continuously during the whole process.

Poly-lysine is formed from lysine in a polycondensation reaction in which water is released when an amino group of one lysine molecule and a carboxyl group of another lysine molecule react with each other to form an amide bond. The process according to the invention requires that water is removed. Any means suitable for removing water from the aqueous lysine solution and/or the reaction mixture may be applied. Water may be removed e.g. by adsorption or by distillation.

The term “about,” as used herein, refers to variation in the numerical quantity that can occur, for example, through typical measuring and handling procedures in the real world; through inadvertent error in these procedures; through differences in the manufacture, source, or purity of the ingredients used to make the compositions or carry out the methods; and the like. The term “reaction temperature” refers to the internal temperature of the reaction mixture in a reaction vessel. The temperature of an external heat source used for heating the reaction vessel may be higher or lower than the reaction temperature.

The aqueous lysine solution and/or the reaction mixture of the invention are part of the “reaction system” which also includes a reaction vessel. The process may be carried out in a continuously or batchwise working reaction system. The process may be carried out in what is called a one-pot mode, in which the lysine is furnished in its entirety in the initial charge and the polycondensation reaction is carried out in a reactor with backmixing. Polycondensation may also be started with only a part of the amount of lysine desired to be furnished in the whole process, wherein the rest of the lysine may be feeded during the polycondensation process batchwise or continuously. Any suitable reaction system may be used such as multistage reactor, a stirred-tank reactor, or a tube reactor. The type of reaction vessel or reactor used, its volume, its isolation measures and other characteristics as well as the actual volume of the reaction mixture in the vessel, have to be recognized during operation accordingly. The one skilled in the art is familiar with the handling of different reactors.

The term “reaction mixture” herein comprises the aqueous lysine solution and/or possible impurities of the same and/or poly-lysine and/or poly-lysine derivative and/or water and/or non-reacted compounds including but not limited to alkenyl-carboxyl acid and/or by-products of the reactions taking place and/or one or more catalysts.

Step (a):

“Aqueous lysine solution” herein means any aqueous lysine-comprising solution such as fermentation broth comprising lysine. Aqueous lysine solution may also mean that lysine in its solid state has been dissolved in a liquid medium comprising water.

Aqueous lysine solutions of the invention may comprise lysine in amounts of at least 5% by weight, at least 10% by weight, at least 20% by weight, at least 30% by weight, at least 40% by weight, at least 50% by weight, at least 60% by weight, at least 70% by weight, at least 75% by weight, at least 80% by weight, at least 85% by weight, at least 90% by weight, or at least 95% by weight, all relative to the total weight of the aqueous lysine solution. The aqueous lysine solution may comprise L-lysine, D-lysine, or any mixture of L-lysine and D-lysine, e.g. a racemic mixture.

Aqueous lysine solution of the invention comprises water in amounts of about 5% by weight, about 10% by weight, about 15% by weight, about 20%, about 25% by weight, about 30% by weight, about 40% by weight, about 50% by weight, about 60% by weight, about 70% by weight, about 80% by weight, about 90% by weight, or about 95% by weight, all relative to the total weight of the aqueous lysine solution.

Aqueous lysine solution of the invention may comprise impurities such as salts originating from the fermentation medium, cell debris originating from the production host cells, metabolites produced by the production host cells during fermentation.

In one embodiment, impurities are comprised in aqueous lysine solution in amounts less than about 20% by weight, less than about 15% by weight, less than about 10% by weight, or less than about 5% by weight, all relative to the total weight of the aqueous lysine solution. “Heating to boiling” means increase of the internal temperature of the aqueous lysine solution to at least about 100° C. In one embodiment heating to boiling includes heating to internal temperatures within the aqueous lysine solution in the range of about 100 to about 110° C., or in the range of about 100° C. to about 105° C.

The pressure within the reaction system may be reduced to about 90 kPa, to about 80 kPa, to about 75 kPa, to about 73 kPa, to about 70 kPa, to about 65 kPa, or to about 60 kPa. The reduction of pressure within a reaction system is usually synonymous with “vacuum is applied”. The boiling temperature usually depends on the actual vacuum applied.

In one embodiment, at least one catalyst may be added to the aqueous lysine solution in step (a) in amounts up to 1% by weight relative the total weight of the reaction mixture. As catalyst sodium hypophosphite may be employed in amounts up to 1% by weight relative the total weight of the reaction mixture.

Step (b):

To start the actual polycondensation reaction, the internal temperature of the reaction mixture is increased to a temperature above boiling temperature, which ranges from about 105° C. to about 180° C. The internal temperature of the reaction mixture may be increased to a temperature in the range of 105° C. to 180° C., in the range of about 135° C. to about 180° C., or in the range of 140° C. to 175° C. In one embodiment, the internal temperature of the reaction mixture is increased to 160° C.

If not done in step (a) already, in one embodiment vacuum is applied in step (b). In one embodiment, pressure within the reaction system has been reduced to a certain extent in step (a) and is further reduced in step (b). The pressure may be reduced as much as for the given reaction system feasible by taking into account that foaming of the reaction mixture has to be avoided. Pressure within the reaction system may be reduced to about 90 kPa, to about 80 kPa, to about 75 kPa, to about 73 kPa, to about 70 kPa, to about 65 kPa, or to about 60 kPa. In one embodiment, vacuum is applied within short time.

In one embodiment, the increase of the internal temperature of the reaction mixture is achieved within short time.

“Within short time” in the context of applying vacuum and/or increase of internal temperature means that the desired pressure reduction and/or increase of the internal temperature of the reaction mixture is achieved within a time-span that is reasonably short for the given reaction system. “Within short time” may mean within ≤1.5 hours, within ≤1 hour, within ≤35 minutes, or within ≤15 minutes.

In one embodiment, pressure within the reaction system is reduced to about 78 kPa within 35 minutes.

Step (c):

If not done in step (a) or (b) already, vacuum may be applied in step (c). In one embodiment, pressure within the reaction system has been reduced to a certain extent in step (a) and/or step (b) and is further reduced in step (b). The pressure may be reduced as much as for the given reaction system feasible by taking into account that foaming of the reaction mixture has to be avoided. Pressure within the reaction system may be reduced to about 90 kPa, to about 80 kPa, to about 75 kPa, to about 73 kPa, to about 70 kPa, to about 65 kPa, or to about 60 kPa. In one embodiment, vacuum is applied within short time.

In one embodiment, pressure has already been reduced in step (b) and is further reduced in step (c). For example, pressure may have been reduced within the reaction system in step (b) to 78 kPa within short time and may be further reduced to 73 kPa in step (c) within short time such as 35 minutes.

The desired internal temperature of the reaction mixture once achieved, is kept until
i. melt viscosity of the reaction mixture in the range of about 350 mPa*s to about 6,500 mPa*s is achieved when measured at 160° C., and
ii. an amine number in the range of about 100 mg KOH/g to about 500 mg KOH/g is achieved.

The melt viscosity to be achieved may be in the range of about 350 mPa*s to about 6,500 mPa*s, or in the range of about 1,000 mPa*s to about 6,500 mPa*s when measured at 160° C. The melt viscosity to be achieved may be in the range of 1000 mPa*s to 6,500 mPa*s, in the range of about 3,000 mPa*s to about 6,500 mPa*s, in the range of about 3,500 mPa*s to about 6,500 mPa*s, in the range of about 4,500 mPa*s to about 6,500 mPa*s, or in the range of about 4,500 mPa*s to about 6,200 mPa*s, or in the range of about 5,000 mPa*s to about 6,200 mPa*s, when measured at 140° C.

The melt viscosity values are those determined at 140° C. or 160° C. For the purposes of this invention, the melt viscosity values are determined by melt rheology measurement (plate-plate) using an I.C.I. Cone Plate Viscosimeter from Epprecht GmbH (now Brookfield GmbH). Said melt rheology measurement is to be performed according to DIN 53018.

The amine number to be achieved may be in the range of 100 KOH/g to 500 mg KOH/g, 100 KOH/g to 400 mg KOH/g, in the range of 150 KOH/g to 450 mg KOH/g, in the range of 150 mg KOH/g to 350 mg KOH/g, in the range of 200 KOH/g to 400 mg KOH/g, in the range of 300 KOH/g to 450 mg KOH/g, or in the range of 350 KOH/g to 400 mg KOH/g.

For the purposes of this invention, the amine number is determined by potentiometric titration of the reaction mixture at 20° C. and 101.3 kPa with trifluoromethanesulfonic acid: amount of KOH in mg equals 1 g amine-comprising substance.

In one embodiment, the desired internal temperature of the reaction mixture once achieved, is kept until
i. melt viscosity of the reaction mixture in the range of about 350 mPa*s to about 6,500 mPa*s is achieved, and
ii. an amine number in the range of about 150 mg KOH/g to about 500 mg KOH/g is achieved.

In one embodiment, the reaction mixture is kept at its internal temperature until a K-value of 11-15 or 12-15 is achieved. The reaction mixture may be kept at its internal temperature until a K-value of 11-14, 12-14, 11-13, or 12-13 is achieved.

The K-values are those determined by measurement of kinematic viscosity via Ubbelohde-viscosimeter (DIN 51562-3) at 20° C. and 101.3 kPa.

The end point of the condensation reaction may also be determined via NIR (near infrared) measurement. For this method the amine number which may be determined according to DIN 53176 or the viscosity measurement which may be determined according to DIN 51562-3 is correlated with NIR spectrum followed by subsequent statistical analysis.

The poly-lysine molecule may have a weight-average molecular weight in the range of about 6,000 g/mol to about 30,000 g/mol, in the range of about 6,000 g/mol to about 23,000 g/mol, in the range of about 8,000 g/mol to about 23,000 g/mol, in the range of about 8,000 g/mol to about 20,000 g/mol, in the range of about 8,000 g/mol to about 17,000 g/mol, in the range of about 10,000 g/mol to about 18,000 g/mol, in the range of about 10,000 g/mol to about 17,000 g/mol, or in the range of about 13,000 g/mol to about 17,000 g/mol.

Weight-average molecular weight for the purposes of this invention is to be determined by size exclusion chromatography (SEC or GPC) using hexafluoro iso-propanol with 0.055% of trifluoro acetic acid potassium salt as an eluent at 35° C. Signal detection is performed by UV/Vis and refractive index sensors.

The poly-lysine molecule may have a polydispersity index of 5.5, of 4.7, of 4.5, of 4, of 3.9, or 3.5. The poly-lysine molecule may have a polydispersity index in the range of 2.0 to 4.4, in the range of 2.0 to 4.0, in the range of 2.6 to 3.9, in the range of 2.3 to 3.5, or in the range of 2 to 3.5.

In one embodiment, the poly-lysine molecule has a weight-average molecular weight in the range of about 6,000 g/mol to about 30,000 g/mol and a polydispersity index of 5.5, or of 4.5.

Step (d):

Depending on the reaction system used, release of the vacuum applied in steps (a), (b), or (c) may be necessary due to adding further reactants such as alkyl-carboxylic acid or alkenyl-carboxylic acid as described in step (e). Release of vacuum may mean that pressure is increased to about 101.3 kPa.

In one embodiment, the poly-lysine obtained is non-modified poly-lysine which is further processed in step (e).

The melt viscosity of non-modified poly-lysine may be in the range of 500 mPa*s to 3,000 mPa*s, or in the range of about 1,000 mPa*s to about 2,300 mPa*s when measured at 160° C. The melt viscosity of non-modified poly-lysine may be in the range of 3,000 mPa*s to 6,500 mPa*s, or in the range of about 3,200 mPa*s to about 6,400 mPa*s when measured at 140° C. In one embodiment, the poly-lysine obtained is modified prior to step (e) by alkoxylation such as ethoxylation (resulting in ethoxylated amine groups) and/or reaction with monofunctional molecules such as amines, isocyanate, carboxylic acids, alcohols such as mPEG, thiols, esters, acid chlorides, anhydrides, and carbonates. The poly-lysine obtained is modified prior to step (e) may be called modified poly-lysine herein.

The melt viscosity of modified poly-lysine, e.g. poly-lysine-mPEG may be in the range of about 350 mPa*s to about 6,500 mPa*s, or in the range of about 350 mPa*s to about 1,000 mPa*s when measured at 160° C. The melt viscosity of modified poly-lysine, e.g. poly-lysine-mPEG may be in the range of about 1,000 mPa*s to about 6,500 mPa*s, or in the range of about 1,000 mPa*s to about 2,000 mPa*s when measured at 140° C.

Step (e):

Alkyl-carboxylic acid or alkenyl-carboxylic acid is added in amounts in the range of 2.5 mol % to 10 mol %, relative to the theoretical amount of non-modified poly-lysine and/or modified polylysine. The amount of alkyl-carboxylic acid or alkenyl-carboxylic acid added may be in the range of 3 mol % to 8 mol %, or about 5 mol %, all relative to the theoretical amount of non-modified poly-lysine and/or modified poly-lysine comprised in the reaction mixture.

Calculation of molar ratio oleic acid as exemplified for addition of 5 mol % of oleic acid

$$n ( Oleic acid ) = 0.05 * n ( poly \text{-} lysine ) = \frac{m ( poly \text{-} lysine )}{M ( lvsine ) - M ( water )}$$

$$molar ratio ( oleic acid ) [ mol \% ] = \frac{n ( Oleic acid )}{n ( polv \text{-} lvsine )} * 100$$

The mass of non-modified or modified poly-lysine is calculated from the amount of reaction water to be removed from the reaction mixture. “Reaction water” means the amount of water that evolves from the polymerization reaction.

The addition of alkyl-carboxylic acid or alkenyl-carboxylic acid is to be conducted “within short time”. In any case this relates to avoidance of reduction of the internal temperature of the reaction mixture as far as possible. “Within short time” in the context of adding alkyl-carboxylic acid or alkenyl-carboxylic acid may mean, that the time-span of supplementation should be kept reasonably short for the given reaction system, e.g. by direct feed of the whole volume alkyl-carboxylic acid or alkenyl-carboxylic acid into the reaction mixture. “Within short time” in the context of adding alkyl-carboxylic acid or alkenyl-carboxylic acid may also mean, that the time-span during which vacuum is released for the purposes of addition of alkyl-carboxylic acid or alkenyl-carboxylic acid is kept reasonably short for the given reaction system. “Within short time” may mean within about 30 minutes, within about 20 minutes, or within about 10 minutes or less.

Alkyl-carboxylic acid may be C₈-C₂₂ or C₁₂-C₁₈ saturated carboxylic acids.

Alkenyl-carboxylic acid may be selected from C₁₆-C₂₂ mono-, and poly-unsaturated fatty acids. Alkyl-carboxylic acid or alkenyl-carboxylic acid may be oxidized to a certain extent, meaning that this oxidation is naturally occurring by exposure to air. These oxidations may be initiated by e.g. oxygen, ozone and nitrous oxide. Oxidized to a certain extent in this context means, that ≤75% of the oleic acid is oxidized. Oxidized to a certain extent may mean, that ≤70%, ≤65%, ≤60%, ≤55%, ≤50%, ≤45%, or ≤40% oleic acid is oxidized.

In one embodiment, the alkyl-carboxylic acid is lauric acid. Lauric acid for supplementation can be derived from animal or plant origin and constitutes a variety of carbon chain lengths, the predominant being the C₁₂ saturated carboxylic acid. Lauric acid is a major component of coconut oil and palm kernel oil.

Lauric acid may be oxidized to a certain extent. Oxidized to a certain extent in this context means, that ≤75% of the lauric acid is oxidized. Oxidized to a certain extent may mean, that ≤70%, ≤65%, ≤60%, ≤55%, ≤50%, ×45%, or ≤40% lauric acid is oxidized.

In one embodiment, the alkenyl-carboxylic acid is oleic acid. Oleic acid for supplementation can be derived from animal or plant origin and constitutes a variety of carbon chain lengths, the predominant being the C₁₈ mono- and poly-unsaturated oleic acid. In one embodiment, oleic acid comprises C₁₈ mono-unsaturated oleic acid in amounts of at least 50%. Oleic acid may comprise C₁₈ mono-unsaturated oleic acid in amounts of at least 55%, at least 60%, at least 65%, at least 70%, at least 75%, or at least 80%.

Oleic acid may be oxidized to a certain extent. Oxidized to a certain extent in this context means, that ≤75% of the oleic acid is oxidized. Oxidized to a certain extent may mean, that ≤70%, ≤65%, ≤60%, ≤55%, ≤50%, ≤45%, or ≤40% oleic acid is oxidized.

Step (f):

If the internal temperature has dropped during addition of alkyl-carboxylic acid or alkenyl-carboxylic acid, the reaction mixture needs again to be increased to the desired internal temperature of the reaction mixture. In one embodiment, this increase is done within short time.

In case, vacuum has been released from the reaction system prior to addition of alkyl-carboxylic acid or alkenyl-carboxylic acid, vacuum may again be applied, meaning that pressure may be reduced to about 90 kPa, to about 80 kPa, to about 75 kPa, to about 73 kPa, to about 70 kPa, to about 65 kPa, or to about 60 kPa. In one embodiment, vacuum is applied within short time. “Within short time” in the context of applying vacuum and/or increase of internal temperature means that the desired pressure reduction and/or increase of the internal temperature of the reaction mixture is achieved within a time-span that is reasonably short for the given reaction system. “Within short time” may mean within ≤1.5 hours, within ≤1 hour, within ≤30 minutes, or within ≤15 minutes.

The desired internal temperature is kept until the number of free alkyl-carboxylic acid or alkenyl-carboxylic acid is 9% by weight, relative to the total weight of poly-lysine derivative. The desired internal temperature may be kept until the number of free acid is less ≤8% by weight, ≤5% by weight, ≤2.7% by weight, or ≤2.5% by weight, all relative to the total weight of the reaction mixture.

For the purpose of this invention, free acid is determined by reacting free alkyl-carboxylic acid or alkenyl-carboxylic acid with MSTFA (N-Methyl-N-(trimethylsilyl)trifluoroacetamide) and detecting the resulting alkyl-carboxylic acid or alkenyl-carboxylic acid silyl ester by gas chromatography. The total amount of free alkyl-carboxylic acid or alkenyl-carboxylic acid is determined by adding commercially available standards and by supplementation of alkyl-carboxylic acid or alkenyl-carboxylic acid. The amount of free alkyl-carboxylic acid or alkenyl-carboxylic acid is calculated based on the amount of non-reacted C₁₂-saturated fatty acid or non-reacted C₁₈-mono-unsaturated fatty acid. In one embodiment, free lauric acid is determined by this method, wherein the number of free lauric acid is calculated based on the amount of non-reacted C₁₂-saturated lauric acid. In one embodiment, free oleic acid is determined by this method, wherein the number of free oleic acid is calculated based on the amount of non-reacted C₁₈-mono-unsaturated oleic acid.

In one embodiment, the poly-lysine derivative obtained by the inventive process is a non-modified poly-lysine functionalized with alkyl-carboxylic acid or alkenyl-carboxylic acid which might be called non-modified poly-lysine derivative herein. This is the case if the poly-lysine molecule has not been modified prior to step (e). In one embodiment, the non-modified polylysine is functionalized with oleic acid, which may be called poly-lysine oleate herein. In one embodiment, the non-modified poly-lysine is functionalized with lauric acid, which may be called poly-lysine laurate herein.

In one embodiment, the poly-lysine derivative obtained by the inventive process is a modified poly-lysine functionalized with alkyl-carboxylic acid or alkenyl-carboxylic acid. This is the case if the poly-lysine molecule has been modified prior to step (e). Such a product may be called modified poly-lysine derivative herein. In one embodiment, the modified poly-lysine is functionalized with oleic acid, which may be called modified poly-lysine oleate herein. In one embodiment, the modified poly-lysine is functionalized with lauric acid, which may be called modified poly-lysine laurate herein.

The non-modified poly-lysine derivative and/or the modified poly-lysine derivative obtained by the inventive process may comprise unreacted lysine and/or possible impurities of the same and/or non-modified poly-lysine and/or modified poly-lysine and/or non-modified poly-lysine derivative and/or modified poly-lysine derivative and/or non-reacted compounds and/or by-products of the reactions taking place and/or water and/or one or more catalysts.

The poly-lysine derivative of the invention is non-crosslinked. In one embodiment, the non-modified and/or modified poly-lysine derivative of the invention is non-crosslinked.

“Non-crosslinked” means that that there is no deliberate cross-linking in the sense of formation of covalent bounds between single poly-lysine derivative molecules or modified poly-lysine derivative molecules introduced. Therefore, essentially no cross-links are introduced by the process of production as such. Essentially no cross-links may mean that the degree of cross-linking is low, such as below 5%, which might be due to cross-linking substances being present in the reaction mixture as impurities of the aqueous lysine solution, such as arginine.

The poly-lysine derivative obtained by the inventive process may have a weight-average molecular weight in the range of about 20,000 g/mol to about 85,000 g/mol or in the range of about 20,000 g/mol to about 60,000 g/mol. The poly-lysine derivative of the invention may have a weight-average molecular weight in the range of about 30,000 g/mol to about 55,000 g/mol, in the range of about 33,000 g/mol to about 50,000 g/mol, in the range of about 40,000 g/mol to about 55,000 g/mol, or in the range of 40,000 g/mol to 50,000 g/mol. Weight-average molecular weight in this context is to be determined by size exclusion chromatography (SEC or GPC) using hexafluoro iso-propanol as described above.

In one embodiment, the poly-lysine derivative obtained by the inventive process has a polydispersity index in the range of about 3.0 to about 10.0. The poly-lysine derivative of the invention may have a polydispersity index in the range of about 4.0 to about 9.0, in the range of about 4.0 to about 8.0, in the range of about 4.5 to about 7.5, or in the range of about 4.5 to about 7.0. In one embodiment, the non-modified and/or modified poly-lysine derivative obtained by the inventive process has a polydispersity index in the range of about 3.0 to about 10.0. The non-modified and/or modified poly-lysine derivative of the invention may have a polydispersity index in the range of about 4.0 to about 9.0, in the range of about 4.0 to about 8.0, in the range of about 4.5 to about 7.5, or in the range of about 4.5 to about 7.0.

In one embodiment, the non-modified and/or modified poly-lysine derivative obtained by the inventive process has a K-value of 11-17, of 12-17, of 13-17, of 14-16.5, of 14.5-16.5, or of 15-16.5.

In one embodiment, the poly-lysine derivative obtained by this process is water-soluble. In one embodiment, the non-modified and/or modified poly-lysine derivative obtained by this process is water-soluble.

“Soluble in water” herein means that the non-modified poly-lysine derivative and/or the modified poly-lysine derivative of the invention, is soluble in water till its saturation concentration is achieved. The saturation concentration of the non-modified poly-lysine derivative and/or the modified poly-lysine derivative means the concentration where water cannot dissolve any more of the substance at 20° C. and 101.3 kPa. Adding more than this maximum concentration of the non-modified poly-lysine derivative and/or the modified poly-lysine derivative will result in phase separation (e.g. precipitation), meaning that any amount exceeding the maximum concentration will remain undissolved in the water.

In one embodiment, the poly-lysine derivative obtained by the inventive process is further processed by the following additional steps:
(g) vacuum applied is released and the temperature within the reaction mixture is reduced to about 150° C. to 100° C.
(h) water is added to yield a solution comprising about 60 parts of poly-lysine derivative and about 40 parts of water.

In one embodiment, the non-modified and/or modified poly-lysine derivative obtained by the inventive process is further processed by the following additional steps:
(g) vacuum applied is released and the temperature within the reaction mixture is reduced to about 150° C. to 100° C.
(h) water is added to yield a poly-lysine derivative solution comprising about 60 parts of modified poly-lysine derivative and about 40 parts of water.

Step (g):

Release of vacuum usually means that the pressure within the reaction system is increased to atmospheric pressure.

In one embodiment, the non-modified poly-lysine derivative obtained by the inventive process, is modified by alkoxylation such as ethoxylation, resulting in ethoxylated amine groups and/or reaction with monofunctional molecules such as amines, isocyanate, carboxylic acids, alcohols such as mPEG, thiols, esters, acid chlorides, anhydrides, and carbonates.

Step (h):

The product obtained by the inventive process comprising non-modified and/or modified polylysine derivative is dissolved in water. Said product may be called poly-lysine derivative solution. In one embodiment, the poly-lysine derivative solution comprises non-modified and/or modified poly-lysine derivative.

Dissolving in water may be realized at a temperature of the reaction system of 150° C. to 100° C. Water is preferably added in amounts that viscosity of the product obtained by the inventive process is reduced to an extent that allows handling of the liquid product. The poly-lysine derivative solution may comprise about 60 parts of at least one poly-lysine derivative and about 40 parts water. In one embodiment, the poly-lysine derivative solution comprises about 30 parts of at least one poly-lysine derivative and about 70 parts water. The poly-lysine derivative solution may comprise about 60 parts poly-lysine derivative and about 40 parts water. In one embodiment, the poly-lysine derivative solution comprises about 30 parts of the poly-lysine derivative and about 70 parts water.

In one embodiment, the poly-lysine derivative solution comprises about 60 parts non-modified and/or modified poly-lysine derivative and about 40 parts water. In one embodiment, the polylysine derivative solution comprises about 30 parts non-modified and/or modified poly-lysine derivative and about 70 parts water.

In one embodiment, the pH of the poly-lysine derivative solution is adjusted to a value in the range of 7 to 14 with inorganic or organic acids. The pH of the poly-lysine derivative solution may be adjusted to a value in the range of 7 to 13, in the range of 8-13, in the range of 9-13, or in the range of 9-11 with inorganic or organic bases.

The current invention, in one aspect, relates to a non-crosslinked poly-lysine functionalized with oleic acid. In one embodiment, said non-crosslinked poly-lysine functionalized with oleic acid is water-soluble.

In one embodiment, the non-crosslinked poly-lysine functionalized with oleic acid is a non-crosslinked non-modified poly-lysine functionalized with oleic acid. In one embodiment, the non-crosslinked poly-lysine functionalized with oleic acid is a non-crosslinked modified poly-lysine functionalized with oleic acid.

In one embodiment, the non-crosslinked non-modified poly-lysine functionalized with oleic acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates. In one embodiment, the non-crosslinked modified poly-lysine functionalized with oleic acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates.

The non-crosslinked poly-lysine oleate of the invention has a weight-average molecular weight in the range of about 20,000 g/mol to about 60,000 g/mol. The non-crosslinked poly-lysine oleate of the invention may have a weight-average molecular weight in the range of about 35,000 g/mol to about 55,000 g/mol, in the range of about 35,000 g/mol to about 50,000 g/mol, in the range of about 30,000 g/mol to about 55,000 g/mol, or in the range of 40,000 g/mol to 55,000 g/mol.

The non-crosslinked non-modified poly-lysine oleate of the invention has a weight-average molecular weight in the range of about 20,000 g/mol to about 60,000 g/mol. The non-crosslinked non-modified poly-lysine oleate of the invention may have a weight-average molecular weight in the range of about 35,000 g/mol to about 55,000 g/mol, in the range of about 35,000 g/mol to about 50,000 g/mol, in the range of about 30,000 g/mol to about 55,000 g/mol, or in the range of 40,000 g/mol to 55,000 g/mol.

The non-crosslinked modified poly-lysine oleate of the invention has a weight-average molecular weight in the range of about 20,000 g/mol to about 60,000 g/mol. The non-crosslinked modified poly-lysine oleate of the invention may have a weight-average molecular weight in the range of about 35,000 g/mol to about 55,000 g/mol, in the range of about 35,000 g/mol to about 50,000 g/mol, in the range of about 30,000 g/mol to about 55,000 g/mol, or in the range of 40,000 g/mol to 55,000 g/mol.

Weight-average molecular weight in this context is to be determined by size exclusion chromatography (SEC or GPC) using hexafluoro iso-propanol as described above.

In one embodiment, the non-crosslinked poly-lysine oleate has a polydispersity index in the range of about 3.0 to about 10.0. The non-crosslinked poly-lysine oleate of the invention may have a polydispersity index in the range of about 4.0 to about 8.0, in the range of about 4.6 to about 7.5, or in the range of about 4.6 to about 7.0, or in the range of about 4.5 to about 7.5. In one embodiment, the non-crosslinked non-modified poly-lysine oleate has a polydispersity index in the range of about 3.0 to about 10.0. The non-crosslinked non-modified poly-lysine oleate of the invention may have a polydispersity index in the range of about 4.0 to about 8.0, in the range of about 4.6 to about 7.5, or in the range of about 4.6 to about 7.0, or in the range of about 4.5 to about 7.5.

In one embodiment, the non-crosslinked modified poly-lysine oleate has a polydispersity index in the range of about 3.0 to about 10.0. The non-crosslinked modified poly-lysine oleate of the invention may have a polydispersity index in the range of about 4.0 to about 8.0, in the range of about 4.6 to about 7.5, or in the range of about 4.6 to about 7.0, or in the range of about 4.5 to about 7.5.

The current invention, in another aspect, relates to a non-crosslinked poly-lysine functionalized with lauric acid. In one embodiment, said non-crosslinked poly-lysine functionalized with lauric acid is water-soluble.

In one embodiment, the non-crosslinked poly-lysine functionalized with lauric acid is a non-crosslinked non-modified poly-lysine functionalized with lauric acid. In one embodiment, the non-crosslinked poly-lysine functionalized with lauric acid is a non-crosslinked modified polylysine functionalized with lauric acid.

In one embodiment, the non-crosslinked non-modified poly-lysine functionalized with lauric acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates. In one embodiment, the non-crosslinked modified poly-lysine functionalized with lauric acid is modified by alkoxylation such as ethoxylation and/or reactions with monofunctional molecules such as amines, isocyanate, carboxylic acids, alcohols such as mPEG, thiols, esters, acid chlorides, anhydrides, and carbonates.

The non-crosslinked poly-lysine laurate of the invention has a weight-average molecular weight in the range of about 20,000 g/mol to about 60,000 g/mol. The non-crosslinked poly-lysine laurate of the invention may have a weight-average molecular weight in the range of about 30,000 g/mol to about 55,000 g/mol, or in the range of 40,000 g/mol to 55,000 g/mol. The non-crosslinked non-modified poly-lysine laurate of the invention may have a weight-average molecular weight in the range of about 20,000 g/mol to about 85,000 g/mol, in the range of about 20,000 g/mol to about 82,000 g/mol, or in the range of about 20,000 g/mol to about 60,000 g/mol. The non-crosslinked non-modified poly-lysine laurate of the invention may have a weight-average molecular weight in the range of about 30,000 g/mol to about 82,000 g/mol, in the range of about 30,000 g/mol to about 55,000 g/mol, in the range of about 40,000 g/mol to about 82,000 g/mol, or in the range of 40,000 g/mol to 55,000 g/mol.

The non-crosslinked modified poly-lysine laurate of the invention has a weight-average molecular weight in the range of about 20,000 g/mol to about 85,000 g/mol, in the range of about 20,000 g/mol to about 82,000 g/mol, or in the range of about 20,000 g/mol to about 60,000 g/mol. The non-crosslinked modified poly-lysine laurate of the invention may have a weight-average molecular weight in the range of about 30,000 g/mol to about 82,000 g/mol, in the range of about 30,000 g/mol to about 55,000 g/mol, in the range of about 40,000 g/mol to about 82,000 g/mol, or in the range of 40,000 g/mol to 55,000 g/mol.

Weight-average molecular weight in this context is to be determined by size exclusion chromatography (SEC or GPC) using hexafluoro iso-propanol as described above.

In one embodiment, the non-crosslinked poly-lysine laurate has a polydispersity index in the range of about 3.0 to about 10.0. The non-crosslinked poly-lysine laurate of the invention may have a polydispersity index in the range of about 4.0 to about 9.0, in the range of about 4.0 to about 8.0, in the range of about 4.5 to about 7.5, or in the range of about 8.0 to about 9.0. In one embodiment, the non-crosslinked non-modified poly-lysine laurate has a polydispersity index in the range of about 3.0 to about 10.0. The non-crosslinked non-modified poly-lysine laurate of the invention may have a polydispersity index in the range of about 4.0 to about 9.0, in the range of about 4.0 to about 8.0, in the range of about 4.5 to about 7.5, or in the range of about 8.0 to about 9.0.

In one embodiment, the non-crosslinked modified poly-lysine laurate has a polydispersity index in the range of about 3.0 to about 10.0. The non-crosslinked modified poly-lysine laurate of the invention may have a polydispersity index in the range of about 4.0 to about 9.0, in the range of about 4.0 to about 8.0, in the range of about 4.5 to about 7.5, or in the range of about 8.0 to about 9.0.

The poly-lysine derivative obtained by the process of the invention may be called component A herein.

The invention provides a storage-stable solid-based composition comprising
(1) a liquid phase comprising components A and B and one or more salts and optionally component C and optionally at least one additional solvent, wherein component A comprises at least one poly-lysine derivative of the invention, wherein component B comprises at least one solvent in which component A is soluble, wherein component C is selected from at least one additional compound, wherein the additional solvent is immiscible with component B, and wherein component A and at least one salt are soluble in component B, and
(2) component D: comprising at least one solid compound, wherein component D is dispersed in the liquid phase,
   wherein at least one poly-lysine derivative comprised in component A is obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

The storage-stable solid-based composition may be a storage-stable homogenous solid-based composition which is stable at 20° C. and/or 54° C. for 14 days.

In one embodiment, component D is solid at 20° C. and 101.3 kPa and is insoluble in component B.

In one embodiment, one or more salts are soluble in component B at 20° C. and 101.3 kPa until the saturation concentration is achieved.

In one embodiment, one or more salts are soluble in the additional solvent at 20° C. and 101.3 kPa until the saturation concentration is achieved.

In one embodiment at least one salt is soluble in component B and at least one salt is soluble in the additional solvent at 20° C. and 101 kPa until the respective saturation concentration in component B and the additional solvent is achieved.

At least one salt may dissociate in the liquid phase (1) into ions, wherein both, the cation and the anion are solvated, preferably the cation and the anion are hydrophilic.

At least one salt comprised in the liquid phase (1) may dissociate in component B into ions, wherein the cation or the anion is amphiphilic. Preferably, the anion is amphiphilic.

In one embodiment, the storage-stable solid-based composition is a two-phasic system, wherein one phase comprises components soluble and/or miscible with component B, and the other phase comprises component D.

In one embodiment, the storage-stable solid-based composition comprises at least three phases, wherein one phase comprises components soluble and/or miscible with component B, a second phase comprises components soluble in a solvent which is immiscible with component B, and a third phase comprises component D. In such a system, component D may not be soluble in the solvent immiscible with component B.

In one embodiment, the storage-stable solid-based composition of the invention is a storage-stable homogenous solid-based composition.

“Homogenous” compositions usually have the same proportions of its components throughout any given sample out of a bigger volume. Solutions are per definition homogenous. “Homogenous solid-based compositions means compositions comprising solid particles which are essentially homogenously distributed within the overall volume of the composition.

Storage-stable homogenous solid-based compositions may include solid-based compositions in which the once dispersed solid particles which have settled during storage over time, but in which the particles did not significantly increase in particle size and are re-dispersible upon shaking, stirring, action of circulating pump, or similar processes.

Storage-stable homogenous solid-based compositions may include solid-based compositions in which the once dispersed solid particles which have settled during storage over time, but in which the particles did not significantly increase in particle size and are re-dispersible upon shaking, stirring, action of circulating pump, or similar processes.

“Salts” means compounds comprising an anion and a cation to form a neutralized compound. Salts usually dissociate into cation and anion when solvated in a solvent. In one aspect, the anion and/or the cation is hydrophilic. In another aspect, the anion or the cation is amphiphilic. Adding salts (which are often called electrolytes) to compositions comprising solvated non-electrolytes may influence the solubility of the non-electrolytes. This may result in the precipitation of a non-electrolyte due to change of the saturation concentration within the composition.

In one aspect of the invention, at least one salt is soluble in component B until the saturation concentration is achieved. At least one salt soluble in component B and/or a solvent miscible with component B may be selected from ionic agrochemically active compounds.

In one aspect of the invention, at least one salt soluble in component B and/or a solvent miscible with component B is selected from ionic surfactants. Ionic surfactants may be selected from anionic and cationic surfactants.

In one embodiment, at least one ionic surfactant is an anionic surfactant. Anionic surfactant means a surfactant with a negatively charged ionic group. Anionic surfactants include, but are not limited to, surface-active compounds that contain a hydrophobic group and at least one water-solubilizing anionic group, usually selected from sulfates, sulfonate, and carboxylates to form a water-soluble compound.

Anionic surfactants may be compounds of general formulae (Ia) or (Ib), which might be called (fatty) alcohol/alkyl (ethoxy/ether) sulfates [(F)A(E)S] when A⁻ is SO₃⁻, (fatty) alcohol/alkyl (ethoxy/ether) carboxylate [(F)A(E)C] when A⁻ is —RCOO⁻:

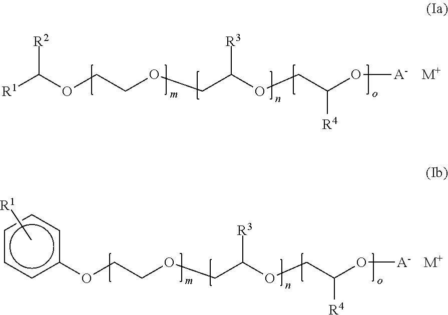

The variables in general formulae (Ia and Ib) are defined as follows:

R¹ is selected from C₁-C₂₃-alkyl (such as 1-, 2-, 3-, 4-C₁-C₂₃-alkyl) and C₂-C₂₃-alkenyl, wherein alkyl and/or alkenyl are linear or branched, and wherein 2-, 3-, or 4-alkyl; examples are n-C₇H₁₅, n-C₉H₁₀, n-C₁₁H₂₃, n-C₁₃H₂₇, n-C₁₅H₃₁, n-C₁₇H₃₅, i-C₉H₁₀, i-C₁₂H₂₅.

R² is selected from H, C₁-C₂₀-alkyl and C₂-C₂₀-alkenyl, wherein alkyl and/or alkenyl are linear or branched.

R³ and R⁴, each independently selected from C₁-C₁₆-alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

A⁻ is selected from —RCOO⁻, —SO₃⁻ and RSO₃⁻, wherein R is selected from linear or branched C₁-C₈-alkyl, and C₁-C₄ hydroxyalkyl, wherein alkyl is.

M⁺ is selected from H and salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine. The integers of the general formulae (Ia) and (Ib) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formulae (Ia) or (Ib) may be of any structure, block copolymers or random copolymers.

Further suitable anionic surfactants include salts (M⁺) of C₁₂-C₁₈ sulfo fatty acid alkyl esters (such as C₁₂-C₁₈ sulfo fatty acid methyl esters), C₁₀-C₁₈-alkylarylsulfonic acids (such as n-C₁₀-C₁₈-alkylbenzene sulfonic acids) and C₁₀-C₁₈ alkyl alkoxy carboxylates.

M⁺ in all cases is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Non-limiting examples of further suitable anionic surfactants include branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, secondary alkanesulfonates (SAS), paraffin sulfonates (PS), sulfonated fatty acid glycerol esters, alkyl- or alkenylsuccinic acid, fatty acid derivatives of amino acids, diesters and monoesters of sulfo-succinic acid.

Anionic surfactants may be compounds of general formula (II), which might be called N-acyl amino acid surfactants:

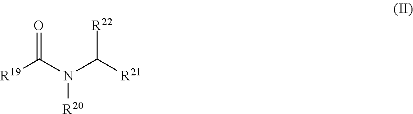

The variables in general formula (II) are defined as follows:

R¹⁹ is selected from linear or branched C₆-C₂₂-alkyl and linear or branched C₆-C₂₂-alkenyl such as oleyl.

R²⁰ is selected from H and C₁-C₄-alkyl.

R²¹ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.

R²² is selected from —COOX and —CH₂SO₃X, wherein X is selected from Li⁺, Na⁺ and K⁺. Non-limiting examples of suitable N-acyl amino acid surfactants are the mono- and dicarboxylate salts (e.g., sodium, potassium, ammonium and ammonium salt of mono-, di, and triethanolamine) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and ammonium salt of mono-, di, and triethanolamine) of

N-acylated alanine, for example, sodium cocoyl alaninate, and triethanolamine lauroyl alaninate; the carboxylate salts (e.g., sodium, potassium, ammonium and ammonium salt of mono-, di, and triethanolamine) of N-acylated glycine, for example, sodium cocoyl glycinate, and potassium cocoyl glycinate; the carboxylate salts (e.g., sodium, potassium, ammonium and ammonium salt of mono-, di, and triethanolamine) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate.

Anionic surfactants may further be selected from the group of soaps. Suitable are salts (M⁺) of saturated and unsaturated C₁₂-C₁₈ fatty acids, such as lauric acid, myristic acid, palmitic acid, stearic acid, behenic acid, oleic acid, (hydrated) erucic acid. M⁺ is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Further non-limiting examples of suitable soaps include soap mixtures derived from natural fatty acids such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

Further non-limiting examples of suitable anionic surfactants include salts (M⁺) of sulfates, sulfonates or carboxylates derived from natural fatty acids such as tallow, coconut oil, palm kernel oil, laurel oil, olive oil, or canola oil. Such anionic surfactants comprise sulfates, sulfonates or carboxylates of lauric acid and/or myristic acid and/or palmitic acid and/or stearic acid and/or oleic acid and/or linoleic acid in different amounts, depending on the natural fatty acids from which the soaps are derived.

In one embodiment, at least one salt is selected from calcium-dodecyl-benzenesulfonate. In one embodiment, the inventive composition comprises two or more different anionic surfactants.

In one embodiment, at least one ionic surfactant is a cationic surfactant. Cationic surfactant means a surfactant with a positively charged ionic group. Typically, these cationic moieties are nitrogen containing groups such as quaternary ammonium or protonated amino groups. The cationic protonated amines can be primary, secondary, or tertiary amines.

Cationic surfactants may be compounds of the general formula (III) which might be called quaternary ammonium compounds (quats):

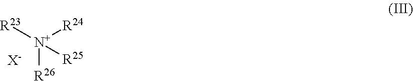

The variables in general formula (III) are defined as follows:

R²³ is selected from H, C₁-C₄ alkyl (such as methyl) and C₂-C₄ alkenyl, wherein alkyl and/or alkenyl is linear or branched.

R²⁴ is selected from C₁-C₄ alkyl (such as methyl), C₂-C₄ alkenyl and C₁-C₄ hydroxyalkyl (such as hydroxyethyl), wherein alkyl and/or alkenyl is linear or branched.

R²⁵ is selected from C₁-C₂₂ alkyl (such as methyl, C₁₈ alkyl), C₂-C₄ alkenyl, C₁₂-C₂₂ alkyl-carbonyloxymethyl and C₁₂-C₂₂ alkylcarbonyloxyethyl (such as C₁₆-C₁₈ alkylcarbonyloxyethyl), wherein alkyl and/or alkenyl is linear or branched.

R²⁶ is selected from C₁₂-C₁₈ alkyl, C₂-C₄ alkenyl, C₁₂-C₂₂ alkyl-carbonyloxymethyl, C₁₂-C₂₂ alkylcarbonyloxyethyl and 3-(C₁₂-C₂₂ alkylcarbonyloxy)-2(C₁₂-C₂₂ alkylcarbonyloxy)-propyl.

X⁻ is selected from halogenid, such as Cl⁻ or Br⁻.

Non-limiting examples of further cationic surfactants include, amines such as primary, secondary and tertiary monoamines with 018 alkyl or alkenyl chains, ethoxylated alkylamines, alkoxylates of ethylenediamine, imidazoles (such as 1-(2-hydroxyethyl)-2-imidazoline, 2-alkyl-1-(2-hydroxyethyl)-2-imidazoline, and the like), quaternary ammonium salts like alkylquaternary ammonium chloride surfactants such as n-alkyl(C₁₂-C₁₈)dimethylbenzyl ammonium chloride, n-tetradecyldimethylbenzylammonium chloride monohydrate, and a naphthylene-substituted quaternary ammonium chloride such as dimethyl-1-naphthylmethylammonium chloride.

Particularly suitable cationic surfactants that may be:
N,N-dimethyl-N-(hydroxy-C₇-C₂₅-alkyl)ammonium salts;
mono- and di(C₇-C₂₅-alkyl)dimethylammonium compounds quaternized with alkylating agents;
ester quats, in particular quaternary esterified mono-, di- and trialkanolamines which are esterified with C₈-C₂₂-carboxylic acids;
imidazoline quats, in particular 1-alkylimidazolinium salts of formulae IV or V

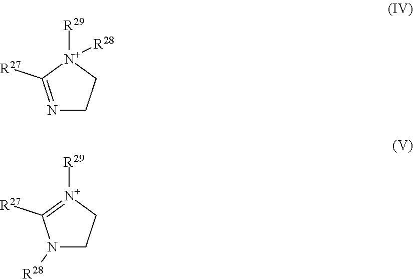

The variables in formulae (IV) and (V) are defined as follows:

R²⁷ is selected from C₁-C₂₅-alkyl and C₂-C₂₅-alkenyl;

R²⁸ is selected from C₁-C₄-alkyl and hydroxy-C₁-C₄-alkyl; R²⁹ is selected from C₁-C₄-alkyl, hydroxy-C₁-C₄-alkyl and a R*—(CO)—R³⁰—(CH₂)ⱼ— radical, wherein R* is selected from C₁-C₂₁-alkyl and C₂-C₂₁-alkenyl; R³⁰ is selected from —O— and —NH—; j is 2 or 3. In one embodiment, the inventive composition comprises two or more different cationic surfactants.

In one aspect of the invention, at least one salt is soluble in a solvent which is immiscible with component B until the saturation concentration is achieved. At least one salt may be selected from ionic agrochemically active compounds soluble in a solvent which is immiscible with component B.

The saturation concentration of a salt is usually the concentration where a salt in a specific solvent or a specific mixture of solvents has reached its maximum concentration dissolvable in the solvent or the mixture of solvents e.g. at 20° C. and 101.3 kPa. Adding more than this maximum concentration of the substance will result in phase separation, meaning that any additional amount of salt exceeding the maximum concentration will remain undissolved in the solvent or mixture of solvents. The actual maximum concentration of salt dissolvable in a solvent usually depends on the solvent used. In one aspect of the invention, solvent means component B herein.

The invention relates to a storage-stable solid-based composition which is a mixture of
(1) a solid based composition comprising at least components A and B and D and optionally component C, wherein component A comprises at least one poly-lysine derivative of the invention, wherein component B comprises at least one solvent in which component A is soluble, and wherein component C is selected from at least one additional compound, and wherein component A is soluble in component B, and
(2) a liquid composition comprising at least (a) one or more salts soluble in component B and (b) at least one solvent which is immiscible with component B and/or (c) at least one solvent which is miscible with component B,
   wherein at least one poly-lysine derivative comprised in component A is obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

The storage-stable solid-based composition may be a storage-stable homogenous solid-based composition which is stable at 20° C. and/or 54° C. for 14 days.

In one embodiment, the liquid composition is liquid at 20° C. and 101.3 kPa.

In one embodiment, one or more salts comprised in the liquid composition (2) are soluble in component B at 20° C. and 101.3 kPa until the saturation concentration is achieved. In one embodiment, one or more salts comprised in the liquid composition (2) are soluble in a solvent which is miscible in with component B at 20° C. and 101.3 kPa until the saturation concentration is achieved.

In one embodiment, one or more salts comprised in the liquid composition (2) are soluble in a solvent which is immiscible in with component B at 20° C. and 101.3 kPa until the saturation concentration is achieved.

In one embodiment at least one salt comprised in the liquid composition (2) is soluble in component B and/or a solvent miscible with component B, and at least one salt is soluble in solvent immiscible with component B at 20° C. and 101 kPa until the respective saturation concentration in component B and the solvent miscible with component B and the solvent immiscible with component B is achieved.

At least one salt comprised in the liquid composition (2) may dissociate in the liquid composition (2) into ions, wherein both, the cation and the anion are solvated, preferably the cation and the anion are hydrophilic.

At least one salt comprised in the liquid composition (2) may dissociate in the liquid composition (2) into ions, wherein the cation or the anion is amphiphilic. Preferably, the anion is amphiphilic. Amphiphilic substances may be called emulsifier herein.

In one embodiment, the storage-stable solid-based composition which is a mixture of the solid based composition (1) and the liquid composition (2) is a two-phasic system, wherein one phase comprises components soluble and/or miscible with component B, and the other phase comprises component D.

In one embodiment, the storage-stable solid-based composition which is a mixture of the solid based composition (1) and the liquid composition (2) comprises at least three phases, wherein one phase comprises components soluble and/or miscible with component B, a second phase comprises components soluble in a solvent which is immiscible with component B, and a third phase comprises component D. In such a system, component D may not be soluble in the solvent immiscible with component B.

In one embodiment, the storage-stable solid-based composition which is a mixture of the solid-based composition (1) and the liquid composition (2) is a storage-stable homogenous solid-based composition.

Component A comprises at least one poly-lysine derivative poly-lysine derivative obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, component A comprises at least one non-modified and/or modified polylysine derivative. In one embodiment, component A comprises at least one non-modified polylysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, component A comprises at least one non-modified and/or modified polylysine oleate. In one embodiment, component A comprises at least one non-modified poly-lysine oleate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, component A comprises at least one non-modified and/or modified polylysine laurate. In one embodiment, component A comprises at least one non-modified polylysine laurate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, component A comprises at least two poly-lysine derivatives selected from the group of non-modified poly-lysine derivative, modified poly-lysine derivative, and non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

Component B comprises at least one compound selected from the group of solvents in which component A is soluble. In one embodiment, component A is soluble in at least one solvent comprised in component B at 20° C. and 101.3 kPa to form a homogenous solution. “Soluble” in solvent herein means that the poly-lysine derivative is soluble in the solvent till the saturation concentration of the poly-lysine derivative is achieved. The saturation concentration of the poly-lysine derivative is usually the concentration where at least one solvent cannot dissolve any more amounts of the poly-lysine derivative at 20° C. and 101.3 kPa. Adding more than this maximum concentration of the substance will result in phase separation (e.g. precipitation), meaning that any amount exceeding the maximum concentration will remain undissolved. Suitable solvents are water, organic solvents such as mineral oil fractions of medium to high boiling point, coal tar oils and oils of vegetable or animal origin, aliphatic, cyclic and aromatic hydrocarbons (e.g. paraffins, tetrahydronaphthalene, alkylated naphthalenes and their derivatives, alkylated benzenes and their derivatives), alcohols, glycols, ketones, fatty acid dimethylamides, fatty acids and fatty acid esters and strongly polar solvents.

In one embodiment, the solvents comprised in component B are miscible with each other. Miscible with each other means, that no phase separation takes place between the solvents mixed. In one embodiment, at least one non-modified and/or modified poly-lysine derivative comprised in component A is soluble in at least one solvent comprised in component B to form a homogenous solution at 20° C. and 101.3 kPa. In one embodiment, at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates comprised in component A is soluble in at least one solvent comprised in component B to form a homogenous solution at 20° C. and 101.3 kPa.

In one embodiment, component B comprises a mixture of two or more solvents, wherein at least one poly-lysine derivative comprised in component A is soluble in the mixture of the two or more solvents to form a homogenous solution at 20° C. and 101.3 kPa.

In one embodiment, at least one non-modified and/or modified poly-lysine derivative comprised in component A is soluble in a mixture of two or more solvents comprised in component B to form a homogenous solution at 20° C. and 101.3 kPa. In one embodiment, at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates comprised in component A is soluble in a mixture of two or more solvents comprised in component B to form a homogenous solution at 20° C. and 101.3 kPa.

In one embodiment, at least one solvent is water-miscible. Water-miscible solvents include aprotic polar solvents and protic solvents. Non-limiting examples of aprotic polar solvents include ketones (e.g. cyclohexanone), lactones (e.g. gamma-butyrolactone), lactames (e.g. N-methyl-2-pyrrolidone), nitriles, tertiary carbonic acid amides, sulfoxides, and carbonates. Non-limiting examples of protic solvents include aliphatic alcohols (e.g. ethanol, propanol, butanol, benzyl alcohol and cyclohexanol), glycols, primary and secondary carbonic acid amides. In one embodiment, at least one solvent is water. In one embodiment, component B comprises water and at least one additional solvent which is miscible with water.

The addition of one or more salts being soluble in component B until their saturation concentration is achieved may change the maximum concentration of at least one poly-lysine derivative of the invention in component B. At least one poly-lysine derivative dissolved in component B may result in phase separation (precipitation, flocculation, and/or turbidity) due to addition of one or more salts to a solution comprising at least components A and B. Component B may be water. In one aspect of the invention, at least one poly-lysine derivative remains dissolved in component B in the presence of one or more salts soluble in component B and/or a solvent miscible with component B. In one aspect of the invention, the at least one poly-lysine derivative comprised in a solid-based composition of the invention remains dissolved in component B in the presence of one or more salts soluble in component B and/or a solvent miscible with component B.

In one embodiment, at least one additional compound comprised in component C is selected from the group of preservatives.

Preservatives are usually added to liquid compositions to prevent alterations of said compositions due to attacks from microorganisms. Non-limiting examples of suitable preservatives include (quaternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride (DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.37-trimethyl-1,3,5-triazine-1,3,5(2H,4H,6H)triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione. In one embodiment, at least one additional compound comprised in component C is selected from the group of surfactants.

In one embodiment, component C comprises at least one non-ionic surfactant. Non-ionic surfactant herein means a surfactant that contains neither positively nor negatively charged functional groups. Examples provided below for surfactants of any kind are to be understood to be non-limiting.

Non-ionic surfactants may be compounds of the general formulae (VIa) and (VIb):

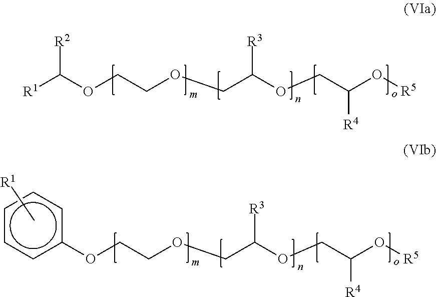

The variables of the general formulae (VIa) and (VIb) are defined as follows: R¹ is selected from C₁-C₂₃ alkyl and C₂-C₂₃ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₂₀ alkyl and C₂-C₂₀ alkenyl, wherein alkyl and/or alkenyl are linear or branched.

R³ and R⁴, each independently selected from C₁-C₁₆ alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.

R⁵ is selected from H and C₁-C₁₈ alkyl, wherein alkyl is linear or branched.

The integers of the general formulae (VIa) and (VIb) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

The non-ionic surfactants of the general formula (VI) may be of any structure, is it block or random structure, and is not limited to the displayed sequence of formula (I).

Non-ionic surfactants may further be compounds of the general formula (VII), which might be called alkyl-polyglycosides (APG):

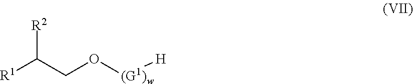

The variables of the general formula (VII) are defined as follows:

R¹ is selected from C₁-C₁₇ alkyl and C₂-C₁₇ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.

R² is selected from H, C₁-C₁₇ alkyl and C₂-C₁₇ alkenyl, wherein alkyl and/or alkenyl are linear or branched.

G¹ is selected from residues of monosaccharides with 4 to 6 carbon atoms, such as glucose and xylose.

The integer w of the general formula (VII) is in the range of from 1.1 to 4, w being an average number.

Non-ionic surfactants may further be compounds of general formula (VIII):

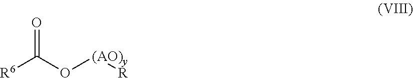

The variables of the general formula (VIII) are defined as follows:

AO is selected from ethylene oxide (EO), propylene oxide (PO), butylene oxide (BO), and mixtures thereof.

R⁶ is selected from C₅-C₁₇ alkyl and C₅-C₁₇ alkenyl, wherein alkyl and/or alkenyl are linear or branched.

R⁷ is selected from H, C₁-C₁₈-alkyl, wherein alkyl is linear or branched.

The integer y of the general formula (VIII) is a number in the range of 1 to 70, preferably 7 to 15. Non-ionic surfactants may further be selected from sorbitan esters and/or ethoxylated or propoxylated sorbitan esters. Non-limiting examples are products sold under the trade names SPAN and TWEEN.

Non-ionic surfactants may further be selected from alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

In one embodiment, at least one non-ionic surfactant is selected from castor oil ethoxylate. In one embodiment of the invention, component C comprises two or more different non-ionic surfactants.

In one embodiment, component C comprises at least one amphoteric surfactant. Amphoteric surfactants are those, depending on pH, which can be either cationic, zwitterionic or anionic. Amphoteric surfactants may be compounds comprising amphoteric structures of general formula (IX), which might be called modified amino acids (proteinogenic as well as nonproteinogenic):

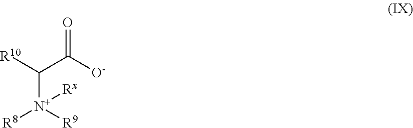

The variables in general formula (IX) are defined as follows:

R⁸ is selected from H, C₁-C₄ alkyl, 02-C₄ alkenyl, wherein alkyl and/or are linear or branched.

R⁹ is selected from C₁-C₂₂-alkyl, C₂-C₂₂-alkenyl, C₁₀-C₂₂ alkylcarbonyl, and C₁₀-C₂₂ alkenylcarbonyl.

R¹⁰ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.

R^{x} is selected from H and C₁-C₄-alkyl.

Amphoteric surfactants may further be compounds comprising amphoteric structures of general formulae (Xa), (Xb), or (Xc), which might be called betaines and/or sulfobetaines:

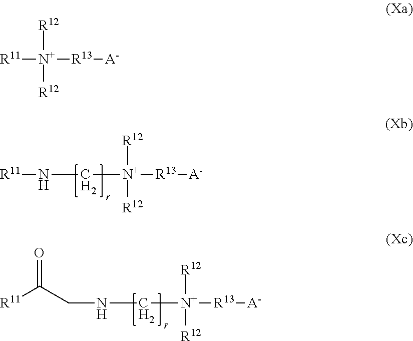

The variables in general formulae (Xa), (Xb) and (Xc) are defined as follows:

R¹¹ is selected from linear or branched C₇-C₂₂ alkyl and linear or branched C₇-C₂₂ alkenyl.

R¹² are each independently selected from linear C₁-C₄ alkyl.

R¹³ is selected from C₁-C₅ alkyl and hydroxy C₁-C₅ alkyl; for example 2-hydroxypropyl.

A⁻ is selected from carboxylate and sulfonate.

The integer r in general formulae (Xa), (Xb), and (Xc) is in the range of 2 to 6.

Amphoteric surfactants may further be compounds comprising amphoteric structures of general formula (VI), which might be called alkyl-amphocarboxylates:

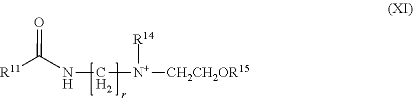

The variables in general formula (XI) are defined as follows:

R¹¹ is selected from C₇-C₂₂ alkyl and C₇-C₂₂ alkenyl, wherein alkyl and/or alkenyl are linear or branched, preferably linear.

R¹⁴ is selected from —CH₂C(O)O⁻M⁺, —CH₂CH₂C(O)O⁻M⁺ and —CH₂CH(OH)CH₂SO₃⁻M⁺. R¹⁵ is selected from H and —CH₂C(O)O⁻

The integer r in general formula (XI) is in the range of 2 to 6.

Non-limiting examples of further suitable alkyl-amphocarboxylates include sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

Amphoteric surfactants may further be compounds comprising amphoteric structures of general formula (XII), which might be called amine oxides (AO):

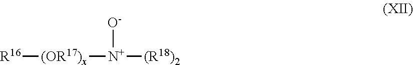

The variables in general formula (XII) are defined as follows:

R¹⁶ is selected from C₈-C₁₈ linear or branched alkyl, hydroxy C₈-C₁₈ alkyl, acylamidopropoyl and C₈-C₁₈ alkyl phenyl group; wherein alkyl and/or alkenyl are linear or branched.

R¹⁷ is selected from C₂-C₃ alkylene, hydroxy C₂-C₃ alkylene, and mixtures thereof.

R¹⁸: each residue can be independently selected from C₁-C₃ alkyl and hydroxy C₁-03; R¹⁵ groups can be attached to each other, e.g., through an oxygen or nitrogen atom, to form a ring structure.

The integer x in general formula (XII) is in the range of 0 to 5, preferably from 0 to 3, most preferably 0.

Non-limiting examples of further suitable amine oxides include C₁₀-C₁₈ alkyl dimethyl amine oxides and C₈-C₁₈ alkoxy ethyl dihydroxyethyl amine oxides. Examples of such materials include dimethyloctyl amine oxide, diethyldecyl amine oxide, bis-(2-hydroxyethyl)dodecyl amine oxide, dimethyldodecylamine oxide, dipropyltetradecyl amine oxide, methylethylhexadecyl amine oxide, dodecylamidopropyl dimethyl amine oxide, cetyl dimethyl amine oxide, stearyl dimethyl amine oxide, tallow dimethyl amine oxide and dimethyl-2-hydroxyoctadecyl amine oxide.

A further example of a suitable amine oxide is cocamidylpropyl dimethylaminoxide, sometimes also called cocamidopropylamine oxide.

In one embodiment, component C comprises two or more different amphoteric surfactants.

In one embodiment, at least one additional compound comprised in component C is selected from the group of foam-controlling substances. Foam-controlling substances include defoamers and foam stabilizers.

Non-limiting examples of suitable defoamers include alkyl phosphates, silicones and such as silicone emulsions (Wacker SRE-PFL, Silikon SRE, from Wacker Chemic, Germany or Rhodorsil from Rhodia, France), long-chain alcohols, fatty acids, salts of fatty acids, defoamers of the type of aqueous wax dispersions, solid defoamers (so-called compounds), organofluorine compounds, and mixtures thereof.

Suitable foam stabilizers include but are not limited to alkanolamides and alkylamine oxides. In one embodiment, at least one additional compound comprised in component C is an antifreeze. An antifreeze usually lowers the freezing point of an aqueous liquid. Non-limiting examples of suitable antifreeze agents include liquid polyols, such as ethylene glycol, propylene glycol and glycerol.

In one embodiment, at least one additional compound comprised in component C is a rheology modifier. Rheology modifiers may be called structuring agents or structurants and may be selected from the following:

i.) Polymeric Structuring Agents

Non-limiting examples of naturally derived polymeric structurants include hydroxyethyl cellulose, hydrophobically modified hydroxyethyl cellulose, carboxymethyl cellulose, polysaccharide derivatives, and mixtures thereof. Suitable polysaccharide derivatives include but are not limited to pectine, alginate, arabinogalactan (gum Arabic), carrageenan, gellan gum, xanthan gum, guar gum and mixtures thereof.

Non-limiting examples of synthetic polymeric structurants include: polycarboxylates, polyacrylates, hydrophobically modified ethoxylated urethanes, hydrophobically modified non-ionic polyols and mixtures thereof. A polycarboxylate polymer may for example be polyacrylate, polymethacrylate or mixtures thereof. The polyacrylate may be for example a copolymer of unsaturated mono- or di-carbonic acid and C₁-C₃₀ alkyl ester of the (meth)acrylic acid.

ii.) Di-Benzylidene Polyol Acetal Derivative

A composition according to the invention may comprise one or more dibenzylidene polyol acetal derivatives (DBPA). The DBPA derivative may comprise a dibenzylidene sorbitol acetal derivative (DBS). Said DBS derivative may be selected from the group consisting of: 1,3:2,4-di-benzylidene sorbitol; 1,3:2,4-di(p-methylbenzylidene) sorbitol; 1,3:2,4-di(p-chlorobenzylidene) sorbitol; 1,3:2,4-di(2,4-dimethyldibenzylidene) sorbitol; 1,3:2,4-di (p-ethyl-benzylidene) sorbitol; 1,3:2,4-di(3,4-dimethyldibenzylidene) sorbitol; and mixtures thereof.

iii.) Di-Amido-Gellants

In one aspect, the external structuring system may comprise a di-amido gellant having a molecular weight from about 150 g/mol to about 1,500 g/mol, or even from about 500 g/mol to about 900 g/mol. Such di-amido gellants may comprise at least two nitrogen atoms, wherein at least two of said nitrogen atoms form amido functional substitution groups. In one aspect, the amido groups are different. In another aspect, the amido functional groups are the same. The di-amido gellant has the following formula:

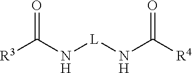

wherein the variables of the di-amido gellant in the above formula are defined as follows:

R³ and R⁴ is an amino functional end-group, or even amido functional end-group, in one aspect

R³ and R⁴ may comprise a pH-tunable group, wherein the pH-tunable amido-gellant may have a pKa of from about 1 to about 30, or even from about 2 to about 10. In one aspect, the pH tunable group may comprise a pyridine. In one aspect, R³ and R⁴ may be different. In another aspect, R³ and R⁴ may be the same.

L is a linking moiety of molecular weight from 14 to 500 g/mol. In one aspect, L may comprise a carbon chain comprising between 2 and 20 carbon atoms. In another aspect, L may comprise a pH-tunable group. In one aspect, the pH-tunable group is a secondary amine. In one aspect, at least one of R³, R⁴ or L may comprise a pH-tunable group.

iv.) Bacterial Cellulose

The term “bacterial cellulose” encompasses any type of cellulose produced via fermentation of a bacteria of the genus Acetobacter such as CELLULON® by CPKelco U.S. and includes materials referred to popularly as microfibrillated cellulose, reticulated bacterial cellulose, and the like.

In one aspect, said fibres may have cross sectional dimensions of 1.6 nm to 3.2 nm by 5.8 nm to 133 nm. Additionally, the bacterial cellulose fibres may have an average microfibre length of at least about 100 nm, or from about 100 to about 1,500 nm. In one aspect, the bacterial cellulose microfibres may have an aspect ratio, meaning the average microfibre length divided by the widest cross sectional microfibre width, of from about 100:1 to about 400:1, or even from about 200:1 to about 300:1.

In one aspect of the invention, the bacterial cellulose is at least partially coated with a polymeric structuring agents (see i. above). In one aspect, the at least partially coated bacterial cellulose comprises from about 0.1% to about 5% w/w, or even from about 0.5% to about 3% w/w of bacterial cellulose; and from about 10% to about 90% w/w of a polymeric structuring agent relative to the total weight of the liquid composition. Suitable bacterial cellulose may include the bacterial cellulose described above and suitable polymeric structuring agents include carboxymethylcellulose, cationic hydroxymethylcellulose, and mixtures thereof.

v.) Cellulose Fibers Non-Bacterial Cellulose Derived

Cellulosic fibers may be extracted from vegetables, fruits or wood. Commercially available examples are Avicel® from FMC, Citri-Fi from Fiberstar or Betafib from Cosun.

vi.) Non-Polymeric Crystalline Hydroxyl-Functional Materials

In one aspect of the invention, the composition may comprise non-polymeric crystalline, hydroxyl functional structurants. Said non-polymeric crystalline, hydroxyl functional structurants may comprise a crystallizable glyceride which can be pre-emulsified to aid dispersion into the liquid composition.

In one aspect, crystallizable glycerides may include hydrogenated castor oil or “HCO” or derivatives thereof, provided that it is capable of crystallizing in the liquid composition.

In one embodiment, component A remains dissolved in the liquid composition comprising components A, B, and C. Component A remains dissolved according to the invention, when no phase separation (precipitiation, flocculation, gelling turbitity) occurs due to the presence of component C.

In one embodiment, at least one solid compound comprised in component D is selected from at least one filling compound. Filling compound is a solid compound contributing texture of a solid based composition. Fillers are usually inert materials.

In one embodiment, at least one solid compound comprised in component D is selected from at least one pigment. Pigment is a solid compound usually contributing color. Pigments are selected from natural and synthetic pigments.

In one embodiment, at least one pigment is a hiding pigment. Hiding pigments may contribute opaqueness and/or UV protection.

In one embodiment, the solid-based composition of the invention is a painting composition.

In one embodiment, the solid-based composition of the invention is an ink.

In one embodiment, the solid-based composition of the invention is a paper coating.

In one embodiment, at least one solid compound comprised in component D is selected from one or more salts which are insoluble in component B. Component B may be water.

In one embodiment, at least one solid compound comprised in component D is selected from at least one agrochemically active compound, which may be called “pesticides” herein. Storage-stable solid-based compositions of the invention comprising at least one solid pesticide may be called storage-stable solid-based agrochemical formulation herein. The storage-stable solid-based agrochemical formulation may be a storage-stable homogenous solid-based agrochemical formulation which is stable at 20° C. and/or 54° C. for 14 days.

Pesticides may be selected from synthetic pesticides and biopesticides. The skilled worker is familiar with pesticides, which can be found, for example, in the Pesticide Manual, 17th Ed. (2015), The British Crop Protection Council, London. Non-limiting examples of pesticides include, but are not limited to fungicides, insecticides, nematicides, herbicides (algicides, arboricides, graminicides), akaricides, molluskicides, ovicides, rodenticides, safeners and growth regulators.

In one embodiment, component D comprises at least one solid pesticide selected from fungicides and/or insecticides and/or nematicides and/or herbicides and/or akaricides and/or molluskicides and/or ovicides and/or rodenticides and/or safeners and/or growth regulators. In one embodiment, component D comprises at least one solid pesticide selected from fungicides and/or insecticides and/or herbicides.

In one embodiment, component D comprises at least one solid fungicide and/or at least one solid insecticide and/or at least one solid nematicide and/or at least one solid herbicide and/or at least one solid akaricide and/or at least one solid molluskicide and/or at least one solid ovicide and/or at least one solid rodenticide and/or at least one solid safener and/or at least one solid growth regulator.

Non-limiting examples of suitable insecticides include compounds from the class of the carbamates, organophosphates, organochlorine insecticides, phenylpyrazoles, pyrethroids, neonicotinoids, spinosins, avermectins, milbemycins, juvenile hormone analogs, alkyl halides, organotin compounds nereistoxin analogs, benzoylureas, diacylhydrazines, METI acarizides, and insecticides such as chloropicrin, pymetrozin, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or their derivatives. Non-limiting examples of suitable fungicides include compounds from the class of dinitroanilines, allylamines, anilinopyrimidines, antibiotics, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy-(2-amino)pyrimidines, hydroxyanilides, imidazoles, imidazolinones, inorganic substances, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothiolates, phthalamic acids, phthalimides, piperazines, piperidines, propionamides, pyridazinones, pyridines, pyridinylmethylbenzamides, pyrimidinamines, pyrimidines, pyrimidinonehydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyltin compounds, triazines, triazoles. Non-limiting examples of suitable herbicides include compounds from the class of acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyl-triazolinones, sulfonylureas, tetrazolinones, thiadiazoles, thiocarbamates, triazines, triazinones, triazoles, triazolinones, triazolocarboxamides, triazolopyrimidines, triketones, uracils, ureas. Non-limiting examples of suitable growth regulators include abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, triexapac-ethyl, and uniconazole.

“Safener” usually means compounds which are added to reduce or to avoid phytotoxic effects towards specific plants.

In one aspect of the invention, the solid-based agrochemical formulations comprises at least one salt soluble in component B which is selected from fertilizers. Component B may be water. Fertilizer includes organic, inorganic, and synthetic fertilizers that may be applied to soils or plant tissue such as leaves to supply plant nutrients which usually enhance growth of plants. Fertilizers typically provide in varying proportions nitrogen and/or phosphorus and/or potassium and/or calcium and/or magnesium and/or sulfur and/or copper and/or iron and/or manganese and/or molybdenum and/or zinc and/or boron and/or other nutrients. Said nutrients may be provided as water-soluble salts.

However, fertilizers may also be comprised in component D if provided in the form of encapsulated fertilizers such as controlled release fertilizers. For this purpose, fertilizers may be encapsuled in a shell that degrades as a specified rate, or fertilizers are provided in a granulated form from which the fertilizer leaches due to contact with water. At least one solid compound comprised in component D selected from fertilizers may therefore be an encapsuled fertilizer and/or a granulated fertilizer.

In one embodiment, component D comprises at least one solid pesticide and/or at least one solid fertilizer.

In one embodiment, solid-based agrochemical formulations comprise two or more solid pesticides and/or two or more solid fertilizers.

In one aspect of the invention, the solid-based agrochemical formulations comprises at least one solid pesticide in component D which is insoluble in component B and at least one pesticide soluble in component B.

In one aspect of the invention, the solid-based agrochemical formulation comprises at least one solid pesticide component D which is insoluble in component B and a solvent which is immiscible with component B in which component D is essentially not soluble. In one embodiment, a solvent immiscible with component B is emulsifiable in component B.

In one aspect of the invention, the solid-based agrochemical formulation comprises at least one solid pesticide component D which is insoluble in component B and a pesticide dissolved in a solvent which is immiscible with component B in which component D is essentially not soluble. Component D is essentially not soluble in a solvent which is immiscible with component B when at 20° C. and 101.3 kPa component D dissolves in said solvent in amounts less than 10% by weight, relative to the total amount of component D. Component D may be essentially not soluble in a solvent which is immiscible with component B when component D dissolves in said solvent in amounts less than 5% by weight, in amounts less than 3% by weight, or less than 1% by weight, all relative to the total amount of component D, all at 20° C. and 101.3 kPa. Component D may be essentially not soluble in a solvent which is immiscible with component B when less than 100 g, less than 50 g, less than 30 g, or less than 1 g of the respective solid compound is soluble in 1000 g of said solvent at 20° C. and 101.3 kPa.

The solid-based agrochemical formulation comprises at least one solid pesticide in amounts in the range of 0.1 to 80% by weight of relative to the total weight of the agrochemical formulation. The solid-based agrochemical formulation may comprise at least one solid pesticide in amounts in the range of 0.1 to 75% by weight, or in the range of 1% to 75% by weight, all relative to the total weight of the agrochemical formulation.

The solid-based agrochemical formulation comprises a poly-lysine derivative according to the invention in amounts in the range of 0.1% to 40% by weight relative to the total weight of the agrochemical formulation. The solid-based agrochemical formulation may comprise a polylysine derivative according to the invention in amounts in the range of 0.1% to 30% by weight, in the range of 0.1% to 20% by weight, in the range of 0.1% to 15% by weight, or in the range of 0.1% to 10% by weight, all relative to the total weight of the agrochemical formulation. In one embodiment, the solid based agrochemical formulation comprises at least one non-modified and/or modified poly-lysine derivative. In one embodiment, the solid based agrochemical formulation comprises at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the solid based agrochemical formulation comprises at least one non-modified and/or modified poly-lysine oleate. In one embodiment, the solid based agrochemical formulation comprises at least one non-modified poly-lysine oleate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the solid based agrochemical formulation comprises at least one non-modified and/or modified poly-lysine laurate. In one embodiment, the solid based agrochemical formulation comprises at least one non-modified poly-lysine laurate which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates.

In one embodiment, the solid-based agrochemical formulation comprises water in amounts in the range of 1% to 99% by weight relative to the total weight of the agrochemical formulation. The solid-based agrochemical formulation may comprise water in amounts in the range of 10% to 90% by weight, or in the range of 10% to 80% by weight, all relative to the total weight of the agrochemical formulation.

The agrochemical formulation of the invention may further comprise one or more formulation auxiliaries in amounts in the range of 0% to 80% by weight relative to the total weight of the agrochemical formulation. The solid-based agrochemical formulation may comprise one or more formulation auxiliaries in amounts in the range of 0% to 70% by weight, or in the range of 0% to 60% by weight, all relative to the total weight of the agrochemical formulation. Formulation auxiliaries are known to those skilled in the art and may be selected from surface-active substances (such as dispersants, emulsifiers, surfactants, solubilizers, protective colloids, wetters and stickers), solvents, solid carriers, defoamers, preservatives, antifreeze agents, rheology modifiers, colorants, antioxidants, retention enhancers (e.g. Lutensol® ON 60), penetration enhancers, adjuvants, tackifiers or binders (for example for the treatment of seeds) oils, and compatibilizer. The solid-based composition of the invention may comprise defoaming agents. Non-limiting examples of suitable, defoaming agents (also called defoamers) include silicone emulsions known for this purpose (Wacker SRE-PFL, Silikon SRE, from Wacker Chemic, Germany or Rhodorsil from Rhodia, France), long-chain alcohols, fatty acids, salts of fatty acids, defoamers of the type of aqueous wax dispersions, solid defoamers (so-called compounds), organofluorine compounds, and mixtures thereof. The amount of defoamers in a solid-based composition may be in the range of 0.01% to 1% by weight, in the range of 0.01% to 0.8% by weight, or in the range of 0.01% to 0.7% by weight, based on the total weight of the solid-based composition of the invention.

Besides component B comprised in the solid-based composition, the agrochemical formulation may comprise at least one additional solvent (formulation auxiliary). Solvents may be selected from water, organic solvents such as mineral oil fractions of medium to high boiling point, coal tar oils and oils of vegetable or animal origin, aliphatic, cyclic and aromatic hydrocarbons (e.g. paraffins, tetrahydronaphthalene, alkylated naphthalenes and their derivatives, alkylated benzenes and their derivatives), alcohols, glycols, ketones, fatty acid dimethylamides, fatty acids and fatty acid esters and strongly polar solvents.

In one embodiment, component B is water and at least one additional solvent is selected from water and other solvents miscible with component B.

In one embodiment, at least one solid compound comprised in a solid-based agrochemical formulation of the invention is insoluble in the total amount of solvent comprised in the agrochemical formulation. At least one solid compound comprised in a solid-based agrochemical formulation of the invention is insoluble in the total amount of solvent comprised in the agrochemical formulation according to the invention, the respective solid compound is soluble in the total amount of solvents comprised in the agrochemical formulation at 20° C. and 101.3 kPa in amounts less than 10% by weight, relative to the total amount of component D. At least one solid compound of component D may be insoluble in the total amount of solvents comprised in the agrochemical formulation, when the respective solid compound is soluble in the total amount of solvents comprised in the agrochemical formulation in amounts less than 5% by weight, in amounts less than 3% by weight, or less than 1% by weight, all relative to the total amount of component D, all at 20° C. and 101.3 kPa. At least one solid compound comprised in a solid-based agrochemical formulation of the invention is insoluble in the total amount of solvent comprised in the agrochemical formulation according to the invention, when less than 100 g of the respective solid compound is soluble in 1000 g of solvents comprised in the agrochemical formulation at 20° C. and 101.3 kPa. At least one solid compound of component D may be insoluble in the total amount of solvents comprised in the agrochemical formulation, when less than 50 g, less than 30 g, or less than 1 g of the respective solid compound is soluble in 1000 g of solvents comprised in the agrochemical formulation at 20° C. and 101.3 kPa.

In one embodiment, component D comprised in a solid-based agrochemical formulation of the invention remains un-dissolved in the agrochemical formulation. Component D remains undissolved according to the invention, when at least 90% by weight of component D remains solid in the agrochemical formulation, relative to the total weight of component D. Component D also remains un-dissolved according to the invention, when at least 95% by weight, at least 97% by weight, at least 99% by weight, or at least 99.5% by weight of component D remains solid in the agrochemical formulation, relative to the total weight of component D.

The invention relates to a storage-stable solid-based agrochemical formulation which is a mixture of
(1) a solid based composition comprising at least components A and B and D and optionally component C, wherein component A comprises at least one poly-lysine derivative of the invention, wherein component B comprises a compound selected from the group of solvents, wherein component C is selected from at least one additional compound, wherein at least one compound comprised in component D is selected from agrochemically active compounds insoluble in component B, and wherein component A is soluble in component B, and
(2) a liquid composition comprising at least (a) one or more salts and (b) at least one solvent which is immiscible with component B and/or (c) at least one solvent which is miscible with component B,
   wherein the solid based composition (1) and/or the liquid composition (2) comprises at least one agrochemically active compound, and wherein at least one poly-lysine derivative comprised in component A is obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, a solvent immiscible with component B is emulsifiable in component B.

In one embodiment, the liquid composition (2) is liquid at 20° C. and 101.3 kPa.

In one embodiment, one or more salts comprised in the liquid composition (2) are soluble in component B at 20° C. and 101.3 kPa until the saturation concentration is achieved. Component B may be water.

In one embodiment, one or more salts comprised in the liquid composition (2) are soluble in a solvent which is miscible in with component B at 20° C. and 101.3 kPa until the saturation concentration is achieved. Component B may be water.

In one embodiment, one or more salts comprised in the liquid composition (2) are soluble in a solvent which is immiscible in with component B at 20° C. and 101.3 kPa until the saturation concentration is achieved. Component B may be water.

In one embodiment at least one salt comprised in the liquid composition (2) is soluble in component B and/or a solvent miscible with component B, and at least one salt is soluble in solvent immiscible with component B at 20° C. and 101 kPa until the respective saturation concentration in component B and the solvent miscible with component B and the solvent immiscible with component B is achieved. Component B may be water. In one embodiment, a solvent immiscible with component B is emulsifiable in component B.

At least one salt comprised in the liquid composition (2) may dissociate in the liquid composition (2) into ions, wherein both, the cation and the anion are solvated, preferably the cation and the anion are hydrophilic.

At least one salt comprised in the liquid composition (2) may dissociate in the liquid composition (2) into ions, wherein the cation or the anion is amphiphilic. Preferably, the anion is amphiphilic. In one embodiment, the solvent miscible with component B comprised in the liquid composition (2) comprises one or more salts. Component B may be water.

In one embodiment, the storage-stable solid-based agrochemical formulation which is a mixture of the solid based composition (1) and the liquid composition (2) is a two-phasic system, wherein one phase comprises components soluble and/or miscible with component B, and the other phase comprises at least one solid agrochemically active compound (component D).

In one embodiment, the storage-stable solid-based agrochemical formulation which is a mixture of a solid based composition (1) and a liquid composition (2) comprises at least three phases, wherein one phase comprises components soluble and/or miscible with component B, a second phase comprises components soluble in a solvent which is immiscible with component B, and a third phase comprises at least one solid agrochemically active compound (component D). In such a system, component D may not be soluble in the solvent immiscible with component B. In one embodiment, a solvent immiscible with component B is emulsifiable in component B.

In one embodiment, the storage-stable solid-based agrochemical formulation which is a mixture of the solid-based composition (1) and the liquid composition (2) is a storage-stable homogenous solid-based composition.

In one aspect, the liquid composition (2) is a solution, in which all components comprised are dissolved in at least one solvent. At least one solvent may be miscible with component B. In one embodiment, all solvents comprised in the liquid composition are miscible with component B. In another aspect, the liquid composition (2) is an emulsion, in which at least two solvents are present which are immiscible in each other. Such a liquid composition (2) may comprise at least one solvent (first solvent) miscible with component B and at least one solvent (second solvent) which is immiscible with the first solvents and/or component B. In one embodiment, a solvent immiscible with component B is emulsifiable in component B.

In one embodiment, the solid-based composition (1) is a solid based agrochemical formulation comprising at least one agrochemically active compound in component D.

In one embodiment, the liquid composition (2) is an agrochemical solution comprising at least one agrochemically active compound selected from pesticides and fertilizers, wherein the agrochemically active compound is dissolved in at least one solvent which is miscible with component B. The agrochemical solutions may comprise at least one agrochemically active compound selected from pesticides and fertilizers, wherein the agrochemically active compound is dissolved in component B. In one embodiment, component B is water.

In one embodiment, the liquid composition (2) is an agrochemical emulsion comprising at least one agrochemically active compound selected from pesticides and fertilizers, wherein the agrochemically active compound is dissolved in at least one solvent which is immiscible with component B. In one embodiment, the solvent immiscible with component B is emulsifiable in component B. In one embodiment, the agrochemically active compound is dissolved in at least one water-immiscible solvent.

The solid-based agrochemical formulations of the invention are stable during storage, meaning that neither significant increase in particle size of the dispersed solid compound (due to e.g. agglomeration), nor gelling, i.e. a significant increase in viscosity, is observed upon storage. Stability during storage herein may also mean that dispersed solid particles which have settled during storage are re-dispersible. Storage stability may be determined by storing a sample at 54° C. for 14 days (see e.g. CIPAC method MT 46-accelerated storage procedure) and comparing particle sizes before storage with particle sizes after storage.

In one embodiment, the storage-stable solid-based agrochemical formulation comprises a mixture of surfactant, wherein the ratio of nonionic surfactant to anionic surfactant in ration selected from 10:4, 7:3, 5:2, and 3:1.

The invention provides a tank-mix comprising the storage-stable solid-based agrochemical formulation according to the invention, wherein the solid-based agrochemical formulation is diluted with water, which optionally comprises fertilizer. The water may be hard and/or soft water. In one embodiment, the tank-mix comprising fertilizer. In one embodiment, the water comprising fertilizer used for dilution may comprise up to 60% w/w water-soluble fertilizer. The storage-stable agrochemical formulation according to the invention may be diluted with water prior to application in order to prepare the so-called tank mix.

The invention provides a method for production of a solid-based composition of the invention comprising the mixing in no specified order in one or more steps components A, B, optionally C, component D, and one or more salts which are soluble in component B or a solvent miscible with component B. In one embodiment, the pH is adjusted of both, the composition and/or solution comprising component A and the composition and/or solution comprising at least one salt soluble in component B or a solvent miscible with component B, before the composition and/or solution comprising component A and the composition and/or solution comprising at least one salt soluble in component B or a solvent miscible with component B are mixed with each other.

The invention provides a method of producing a storage-stable solid-based composition comprising the steps of
I. providing a solution (1) by dissolving component A in component B, and optionally adjusting to pH of solution (1), and
II. providing a liquid (2) by dissolving one or more salts in at least one solvent, and optionally adjusting the pH of liquid (2), and
III. providing a solid-based composition (3) by dispersing component D in a dispersing medium comprising at least one dispersant in which component D is insoluble, and
IV. mixing at least the solution (1) and the solid-based composition (3) and optionally the liquid (2),
   wherein at least one poly-lysine derivative comprised in component A is obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, the dispersing medium of step (III) comprises component A and component B and optionally component C.

The method of producing a storage-stable solid-based composition of the invention may include the process of comminution, which takes place in step (III). The method of producing a storage-stable solid-based composition may provide a storage-stable homogenous solid-based composition which is stable at 20° C. and/or 54° C. for 14 days.

In one embodiment, the liquid (2) comprises at least one salt dissolved in a solvent miscible with component B. The liquid (2) may be a solution. At least one salt may be selected from agrochemically active compounds which are soluble in component B or in solvents miscible with component B.

In one embodiment, the liquid (2) comprises at least one salt dissolved in a solvent which is miscible with component B, and at least one solvent which is immiscible with component B. In one embodiment, at least one solvent immiscible with component B is emulsifiable with component B. The liquid (2) may be an emulsion. At least one salt may be selected from agrochemically active compounds which are soluble in a solvent immiscible with component B. The storage-stable solid-based composition may be stable at 20° C. and/or 54° C. for 14 days.

The invention provides a method of producing a storage-stable solid-based agrochemical formulation comprising the mixing in no specified order in one or more steps components A, B, optionally C, component D, one or more salts, and at least one formulation auxiliary, wherein component D comprises at least one solid pesticide and/or at least one solid fertilizer. In one embodiment, the method of producing a storage-stable agrochemical formulation of the invention comprises the mixing in no specified order in one or more steps components A, B, optionally C, component D, at least one liquid pesticide and/or liquid fertilizer, and at least one formulation auxiliary, wherein component D comprises at least one solid pesticide and/or at least one solid fertilizer, and wherein at least one liquid pesticide and/or liquid fertilizer is soluble in component B or a solvent miscible with component B. In one embodiment, the method of producing a storage-stable agrochemical formulation comprises the mixing in no specified order in one or more steps of the solid-based composition of the invention, at least one liquid pesticide and/or liquid fertilizer, and at least one formulation auxiliary, wherein the liquid composition comprises at least one solid pesticide and/or at least one solid fertilizer. The method of producing a storage-stable agrochemical formulation may provide a storage-stable solid-based agrochemical formulation which is a storage-stable at 20° C. and/or 54° C. for 14 days. Components A, B, C, D, and formulation auxiliaries are those disclosed above.

The solid-based composition of the invention may be prepared by the process of comminution. Usually comminution processes divide a solid into fine particles in the dispersing medium or in a dry state before mixing with a dispersing medium. The one skilled in the art is familiar with the specifics of wet and dry comminution. The effectiveness of comminution depends on the shape and crystal form of particles. Usually, wet comminution is more effective than dry comminution and reduces particle size better. Wet comminution is often operated by using impeller mills, ball mills, small-media mills (such as sand mills and bead mills), vibratory mills, roll mills or ultrasonic dispersors. Further examples of mills useful include but are not limited to agitator ball mills, circulating mills (agitator ball mills with pin grinding system), disk mills, annular chamber mills, double cone mills, triple roll mills, batch mills, and colloid mills.

To dissipate the heat energy introduced during the comminution process, the comminution chambers may be fitted with cooling systems.

The particle size within 50% of the total amount of solid compound (dx₅₀) comprised in the solid-based composition of the invention may be about ≤50 μm, about ≤30 μm, about ≤20 μm, or about ≤10 μm.

In one embodiment, the particle size within 90% of the total amount of solid compound (dx₉₀) is less than 100 μm, less than 50 μm, less than 30 μm, or less than 20 μm.

Size particle distributions may be measured by any suitable method known to those skilled in the art. Suitable methods include but are not limited to methods using laser diffraction. Descriptions for the use of laser diffraction methods are provided e.g. in ISO 13320-1, CIPAC MT184 (Handbook K).

The invention provides a method for producing of a tank-mix comprising the mixing in no specified order in one or more steps a solid-based agrochemical formulation of the invention and water. In one embodiment, the tank-mix is a spray-mix. The water may be hard and/or soft water.

In one embodiment, the method for producing of a tank-mix comprising the mixing in no specified order in one or more steps a solid-based agrochemical formulation of the invention and water, wherein the water comprises fertilizer. The water may be hard and/or soft water. The water comprising fertilizer may comprise up to 60% w/w water-soluble fertilizer.

The storage-stable agrochemical formulation according to the invention may be diluted with water prior to application in order to prepare the so-called tank mix.

Oils of various types, wetters, adjuvants, herbicides, bactericides, fungicides may be added to the tank mix immediately prior to application (tank mix). These agents can be admixed to the compositions according to the invention in a weight ratio from 1:100 to 100:1, preferably 1:10 to 10:1. The concentration of the agrochemically active compound in the tank mix can be varied within substantial ranges. In general, they are between 0.0001% and 10%, preferably between 0.01% and 1%. When used in plant protection, the application rates may range between 0.01 and 2.0 kg of agrochemically active compound per ha, depending on the nature of the desired effect.

Hard water is usually water that has high mineral content in contrast with soft water. In one embodiment, the mineral content of water ranges within the mineral content of CIPAC B and CIPAC D water. The mineral content may be the one of CIPAC B water or the one of CIPAC D water.

In one embodiment, at least one non-modified and/or modified poly-lysine derivative is used as stabilizing and/or wetting and/or dispersing agent in solid-based compositions.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used as stabilizing and/or wetting and/or dispersing agent in solid-based compositions.

In one embodiment, at least one non-modified and/or modified poly-lysine derivative is used as stabilizing agent in solid-based compositions comprising one or more salts which are dissolved in the dispersing medium, wherein the poly-lysine derivative obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used as stabilizing agent in solid-based compositions comprising one or more salts which are dissolved in the dispersing medium.

In one embodiment, the poly-lysine derivative is used as wetting and/or dispersing agent for solid particles during a comminution process.

In one embodiment, at least one non-modified and/or modified poly-lysine derivative is used as wetting and/or dispersing agent for solid particles during a comminution process.

In one embodiment, at least one non-modified poly-lysine derivative at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used as wetting and/or dispersing agent for solid particles during a comminution process.

In one aspect, the invention relates to a method of stabilizing a solid-based composition comprising the steps of adding to such a composition a poly-lysine derivative obtained by the process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

The method of stabilizing a solid-based composition may provide a storage-stable homogenous solid-based composition which is stable at 20° C. and/or 54° C. for 14 days.

In one embodiment, the invention provides a method of stabilizing a solid-based composition comprising the steps of
(1) providing a solid-based composition comprising at least component D in a liquid dispersing medium comprising at least one solvent miscible with component B, and optionally adjusting the pH of the solid based-composition, and
(2) providing a solution comprising component A dissolved in component B, and optionally adjusting the pH of the solution, and
(3) mixing (1) and (2),
   wherein component D is insoluble in the liquid dispersing medium.

In one embodiment, one or more salts are comprised in solubilized form in the dispersing medium. The dispersing medium and component B may be miscible with each other.

In one embodiment, the solid-based composition is mixed with a liquid phase comprising at least one solvent immiscible with component B, wherein the pH of this liquid phase is adjusted prior to mixing. In one embodiment, a solvent immiscible with component B is emulsifiable in component B. The liquid phase comprising a solvent immiscible with component B may comprise at least one salt which is soluble in at least one solvent immiscible with component B.

In one embodiment, the invention provides a method of stabilizing a solid-based composition comprising the steps of
(1) providing a solid-based composition comprising at least component D in a liquid dispersing medium, wherein the liquid dispersing medium comprises component A dissolved in component B, and optionally adjusting the pH of the solid based-composition, and
(2) providing a solution comprising at least one salt, and optionally adjusting the pH of the solution, and
(3) mixing (1) and (2),
   wherein component D is insoluble in the liquid dispersing medium.

In one embodiment, the solid-based composition is mixed with a liquid phase comprising at least one solvent immiscible with component B, wherein the pH of this liquid phase is adjusted prior to mixing. In one embodiment, a solvent immiscible with component B is emulsifiable in component B. The liquid phase comprising a solvent immiscible with component B may comprise at least one salt which is soluble in at least one solvent immiscible with component B.

In one embodiment, the invention provides a method of stabilizing a solid-based agrochemical formulation comprising the steps of
(1) providing a solid-based composition comprising at least component D in a liquid dispersing medium, and optionally adjusting the pH of the solid based-composition, and
(2) providing a solution comprising component A dissolved in component B, and optionally adjusting the pH of the solution, and
(3) mixing (1) and (2),
   wherein at least one compound comprised in component D is selected from agrochemically active compounds insoluble in the liquid dispersing medium.

In one embodiment, one or more salts are comprised in solubilized form in the dispersing medium. The dispersing medium and component B may be miscible with each other. At least one salt may be selected from agrochemically active compounds soluble and/or miscible with component B, such as glyphosate, glyphosinate, agrochemically active complexing agent (e.g. dithiocarbamate such as mancozeb).

In one embodiment, the solid-based composition is mixed with a liquid phase comprising at least one solvent immiscible with component B, wherein the pH of this liquid phase is adjusted prior to mixing. The liquid phase comprising a solvent immiscible with component B may comprise at least one salt which is soluble in at least one solvent immiscible with component B. At least one salt which is soluble in a solvent immiscible with component B may be selected from agrochemically active compounds. In one embodiment, a solvent immiscible with component B is emulsifiable in component B.

The method of stabilizing a solid-based agrochemical formulation may provide a storage-stable homogenous solid-based agrochemical formulation which is stable at 20° C. and/or 54° C. for 14 days.

In one embodiment, the solid-based agrochemical formulation is a tank-mix.

In one embodiment, the invention provides a method of stabilizing a solid-based composition comprising the steps of
(1) providing a solid-based composition comprising at least component D in a liquid dispersing medium, wherein the liquid dispersing medium comprises component A dissolved in component B, and optionally adjusting the pH of the solid based-composition, and
(2) providing a solution comprising at least one salt, and optionally adjusting the pH of the solution, and
(3) mixing (1) and (2),
   wherein at least one compound comprised in component D is selected from agrochemically active compounds insoluble in the liquid dispersing medium.

In one embodiment, the solid-based composition is mixed with a liquid phase comprising at least one solvent immiscible with component B, wherein the pH of this liquid phase is adjusted prior to mixing. In one embodiment, a solvent immiscible with component B is emulsifiable in component B. The liquid phase comprising a solvent immiscible with component B may comprise at least one salt which is soluble in at least one solvent immiscible with component B.

The current invention relates to the use of or method of use of at least one poly-lysine derivative to increase storage stability of solid-based compositions comprising a dispersing medium comprising at least one solvent miscible with component B and at least one dispersant, and one or more salts which are dissolved in the dispersing medium, and component D, when compared to solid-based compositions lacking said poly-lysine derivative, wherein the poly-lysine derivative is obtained by a process comprising the steps of
(a) heating an aqueous lysine solution to boiling
(b) increasing temperature of the aqueous lysine solution to a reaction temperature in the range of about 105° C. to about 180° C.
(c) keep the reaction temperature in the range of about 105° C. to about 180° C. until
   i. melt viscosity of the reaction mixture in the range of about 350 mPa*s to about 6,500 mPa*s when measured at 160° C. and
   ii. an amine number in the range of about 100 mg KOH/g to about 500 mg KOH/g is achieved
(d) optionally, the vacuum applied is released
(e) add alkyl-carboxylic acid or alkenyl-carboxylic acid in amounts of 2.5 mol % to 10 mol %, relative to the theoretical amount of poly-lysine comprised in the reaction mixture
(f) increase or keep the reaction temperature in the range of about 105° C. to about 180° C. until number of free alkyl-carboxylic acid or alkenyl-carboxylic acid is 9% by weight, relative to the total weight of the reaction mixture,
   wherein vacuum is applied either in step (a), (b) and/or (c) and water is removed continuously during the whole process.

In one embodiment, at least one non-modified poly-lysine derivative and/or modified poly-lysine derivative is used to increase storage stability of solid-based compositions comprising one or more salts which are dissolved in the dispersing medium, when compared to solid-based compositions lacking said non-modified poly-lysine derivative.

In one embodiment, at least one non-modified poly-lysine derivative which has been modified by alkoxylation such as ethoxylation and/or reaction with monofunctional molecules such as amines, isocyanate, carboxylic acids, alcohols such as mPEG, thiols, esters, acid chlorides, anhydrides, and carbonates is used to increase storage stability of solid-based compositions comprising one or more salts which are dissolved in the dispersing medium, when compared to solid-based compositions lacking said non-modified poly-lysine derivative.

In one embodiment, poly-lysine oleate and/or poly-lysine laurate is used to increase storage stability of solid-based compositions comprising one or more salts which are dissolved in the dispersing medium, when compared to solid-based compositions lacking said non-modified poly-lysine derivative.

In one aspect of the invention, the dispersing medium of the solid-based composition comprises a solvent miscible with component B and at least one salt soluble in component B and/or a solvent miscible with component B. Component B may be water.

In one embodiment, the solid-based composition comprises a dispersing medium and at least one additional solvent which is immiscible with the dispersing medium. In one embodiment, a solvent immiscible with the dispersing medium is emulsifiable in the dispersing medium. In one embodiment, the solid-based composition comprises a dispersing medium and at least one salt dissolved in an additional solvent which is immiscible with the dispersing medium.

In one embodiment, the current invention relates to the use of or method of use of at least one poly-lysine derivative to increase storage stability of solid-based agrochemical formulations comprising one or more salts which are dissolved in the dispersing medium, wherein the dispersing medium comprises at least one solvent miscible with component B. At least one salt may be selected from agrochemically active compounds soluble in component B and/or at least one solvent miscible with component B. Component B may be water.

In one embodiment, the solid-based agrochemical formulation comprises a dispersing medium and at least one additional solvent which is immiscible with the dispersing medium. In one embodiment, a solvent immiscible with the dispersing medium is emulsifiable in the dispersing medium. In one embodiment, the solid-based agrochemical formulation comprises a dispersing medium and at least one salt dissolved in an additional solvent which is immiscible with the dispersing medium. At least one salt may be selected from agrochemically active compounds soluble in a solvent immiscible with component B. Component B may be water.

In one embodiment, the solid-based agrochemical formulation is a tank-mix.

The present invention provides the use or method of use of an agrochemical formulation of the invention for the treatment of plants. In one embodiment, an agrochemical formulation according to the invention is used for the treatment of crop plants.

Non-limiting examples of “crop plants”, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sun flowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevie); natural rubber plants or ornamental and forestry plants, such as flowers, shrubs, broad-leaved trees or evergreens, e. g. conifers; and on the plant propagation material, such as seeds, and the crop material of these plants.

The term “crop plant” is to be understood as including plants which have been modified by breeding, mutagenesis or genetic engineering including but not limiting to agricultural biotech products on the market or in development (cf. http://www.bio.org/speeches/pubs/er/agri products. asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties. The use or method of use of agrochemical formulations of the invention may relate to the improvement of health of “crop plants” which may be determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress.

The use or method of use of agrochemical formulations of the invention may relate to the controlling of phytopathogenic fungi and/or undesired plant growth and/or undesired insect or mite attack and/or for regulating the growth of plants, where the agrochemical formulation of the invention is allowed to act on the respective pests, their environment or the plants to be protected from the respective pest, the soil and/or on undesired plants and/or the useful plants and/or their environment.

The invention provides the use or method of use of an agrochemical formulation according to the invention to treat plant propagation material.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring. In one embodiment, treatment of plant propagation materials with the composition and/or agrochemical formulation of the invention is used for controlling a multitude of fungi on cereals, such as wheat, rye, barley and oats; rice, corn, cotton and soybeans.

The invention relates to seed which has been treated with an agrochemical formulation of the invention. The seed may be dressed with the composition and/or agrochemical formulation of the invention. Dressing means that the seed is treated with the composition and or agrochemical formulation and the composition and/or agrochemical formulation remains on the seed. This composition and/or agrochemical composition may be applied to the seed in undiluted or, preferably, diluted form. Here, the composition in question can be diluted 2- to 10-fold, so that from 0.01% to 60% by weight, or from 0.1% to 40% by weight, of pesticide are present in the compositions and/or agrochemical formulation to be used for dressing the seed. The application can take place before sowing.

The treatment of plant propagation material, such as the treatment of seed, is known to the skilled worker and is carried out by dusting, coating, pelleting, dipping or soaking the plant propagation material, the treatment may be effected by pelleting, coating and dusting, so that, for example, premature germination of the seed is prevented. In the treatment of seed, one may use pesticide amounts of from 1 to 1000 g/100 kg, or from 5 to 100 g/100 kg propagation material or seed.

### EXAMPLES

### Example 1—General Process for Synthesis of Poly-Lysine Derivative

| | |
|---|---|
| Initial charge | 500 g Lysine solution 50% in water, 1.25 g sodium |
| | hypophosphite |
| feed 1: | 2000 g Lysine solution 50% in water |
| feed 2: | . . . g alkyl-carboxylic acid or alkenyl-carboxylic acid |

The initial charge is started to be heated. At an internal temperature of 100° C., feed 1 is started to be added to the boiling initial charge. After 45 minutes the internal temperature of 160° C. should be achieved. The internal temperature of the reaction mixture (i.e reaction temperature) is to be kept at this temperature at the following. Feed 1 is added within 5 hours to the reaction mixture.

After having added the whole feed 1, the pressure within the reaction system is to be reduced to 780 mbar within 35 minutes.

Within further 35 minutes, the pressure within the reaction system is to be further reduced to 725 mbar. The reaction mixture is to be kept at 160° C. and 725 mbar for additional 2 hours and 20 minutes.

During the whole time, evaporating water is distilled of.

The K-value is to be checked during the reaction several times. For this purpose, the vacuum is to be released to collect a sample and is to be applied again immediately after collecting the probe.

The K-value is to be determined by measurement of kinematic viscosity via Ubbelohde-viscosimeter (DIN 51562-3).

The amine number is to be checked after achieving the target K-value by potentiometric titration of the reaction mixture at 20° C. and 101.3 kPa with trifluoromethanesulfonic acid: amount of KOH in mg equals 1 g amine-comprising substance.

The molecular weight, viscosity and PDI are determined.

After reaching the target K-value and amine number, vacuum is to be released and feed 2 is to be added to the reaction mixture within 10 minutes.

Immediately after finishing the addition of feed 2, pressure within the reaction system is to be reduced to 725 mbar and the internal temperature of the reaction mixture is to be kept at 160° C. for another 4 hours. During this time, evaporating water is distilled of.

The weight-average molecular weight of the resulting poly-lysine derivative is to be determined by size exclusion chromatography (SEC or GPC) using hexafluoro iso-propanol with 0.055% of trifluoro acetic acid potassium salt as an eluent at 35° C. Signal calibration is done using a PMMA standard from the company PSS with molecular weights from 800 g/mol to 2,200,000 g/mol. Signal detection is performed by UV/Vis and refractive index sensors. Typically, 50 μL of sample having a concentration of 1.5 mg/mL are injected onto the column setup (1^{st} precolumn 8 mm inner diameter, 5 cm length; separation column one 7.5 mm inner diameter, 30 cm length; separation column two 7.5 mm inner diameter, 30 cm length) with a flow rate of 0.85 mL/min. Afterwards the internal pressure is to be set to atmospheric pressure and the temperature is to be reduced to 120° C. The product obtained is diluted with water to a concentration of about 30% and the pH is adjusted with lactic acid to a pH value of about 8.

### Example 2

| | |
|---|---|
| Initial charge | 500 g Lysine solution 50% in water, 1.25 g sodium |
| | hypophosphite |
| feed 1: | 2000 g Lysine solution 50% in water |
| feed 2: | 120.8 g Oleic acid |

The procedure described in example 1 was conducted until the poly-lysine reached a K value of 11; the poly-lysine had a Mw of 6,990 g/mol, Mn of 2,720 g/mol, and a PDI of 2.6. The amine number was 422, melt viscosity 3,280 mPa*s (measured with Epprecht viscosimeter at 140° C.), melt viscosity 1,000 mPa*s (measured with Epprecht viscosimeter at 160° C.).

Then feed 2 was introduced into the reaction mixture as described in example 1; the resulting poly-lysine oleate had a K-value of 14.9, an amine number of 315 mg KOH/g, Mw of 46,200 g/mol, Mn of 6,740 g/mol and a PDI of 6.9. Free acid was 2.1% relative to the total weight of the poly-lysine derivative (solid matter). The pH of the poly-lysine oleate solution was 8.3.

### Example 3

| | |
|---|---|
| Initial charge | 500 g Lysine solution 50% in water, 1.25 g sodium |
| | hypophosphite |
| feed 1: | 2000 g Lysine solution 50% in water |
| feed 2: | 120.8 g Oleic acid |

The procedure described in example 1 was conducted until the poly-lysine reached a K value of 12.3; the poly-lysine had a Mw of 17,100 g/mol, Mn of 4,910 g/mol, and a PDI of 3.5. The amine number was 391, melt viscosity 6,320 mPa*s (measured with Epprecht viscosimeter at 140° C.), melt viscosity 2,240 mPa*s (measured with Epprecht viscosimeter at 160° C.).

Then feed 2 was introduced as described in example 1; the resulting poly-lysine oleate had a K-value of 15.1, an amine number of 321 mg KOH/g, Mw of 49,700 g/mol, Mn of 7,420 g/mol and a PDI of 6.7. The pH of the poly-lysine oleate solution was 8.5.

### Example 4

| | |
|---|---|
| Initial charge | 500 g Lysine solution 50% in water, 1.25 g sodium |
| | hypophosphite |
| feed 1: | 2000 g Lysine solution 50% in water |
| feed 2: | 362.4 g Oleic acid |

The procedure described in example 1 was conducted until the poly-lysine reached a K value of 11; the poly-lysine had a Mw of 12,900 g/mol, Mn of 3,920 g/mol, and a PDI of 3.3. The amine number was 422, melt viscosity 3,280 mPa*s (measured with Epprecht viscosimeter at 140° C.), melt viscosity 1,000 mPa*s (measured with Epprecht viscosimeter at 160° C.).

Then feed 2 was introduced as described in example 1; the resulting poly-lysine oleate had an amine number of 221 mg KOH/g, Mw of 44,000 g/mol, Mn of 6,500 g/mol and a PDI of 6.8. Free acid was 2.4% relative to the total weight of the poly-lysine derivative (solid matter). The pH of the poly-lysine-oleate solution was 8.0.

### Example 5

| | |
|---|---|
| Initial charge | 500 g Lysine solution 50% in water, 1.25 g sodium |
| | hypophosphite |
| feed 1: | 2000 g Lysine solution 50% in water |
| feed 2: | 85.67 g Lauric acid |

The procedure described in example 1 was conducted until the poly-lysine reached a K-value of 12; the poly-lysine had a Mw of 22,700 g/mol, Mn of 5,850 g/mol, and a PDI of 3.9. The amine number was 391, melt viscosity 6,320 mPa*s (measured with Epprecht viscosimeter at 140° C.), melt viscosity 2,240 mPa*s (measured with Epprecht viscosimeter at 160° C.).

Then feed 2 was introduced into the reaction mixture as described in example 1; the resulting poly-lysine laurate had a K-value of 16.2, an amine number of 313 mg KOH/g, Mw of 81,400 g/mol, Mn of 9,340 g/mol and a PDI of 8.7. Free acid was 2.7% relative to the total weight of the poly-lysine derivative (solid matter). The pH of the poly-lysine laurate solution was 8.8.

### Example 6

| | |
|---|---|
| Initial charge | 500 g Lysine solution 50% in water, 1.25 g sodium |
| | hypophosphite, 212.5 g mPEG |
| | (Mw = 5000 g/mol, Pluriol A 5010E) |
| feed 1: | 2000 g Lysine solution 50% in water |
| feed 2: | 120.8 g Oleic acid |

The procedure described in example 1 was conducted until the poly-lysine reached a K-value of 12; the poly-lysine had a Mw of 13,900 g/mol, Mn of 3,000 g/mol, and a PDI of 4.7. The amine number was 395, melt viscosity 1,280 mPa*s (measured with Epprecht viscosimeter at 140° C.), melt viscosity 360 mPa*s (measured with Epprecht viscosimeter at 160° C.).

Then feed 2 was introduced into the reaction mixture as described in example 1; the resulting poly-lysine-oleate-mPEG had a K-value of 16.1, an amine number of 276 mg KOH/g, Mw of 34,400 g/mol, Mn of 7,450 g/mol and a PDI of 4.6. Free acid was 1.8% relative to the total weight of the poly-lysine derivative (solid matter). The pH of the poly-lysine-oleate-mPEG solution was 9.

### Example 7—Comparative Example: Synthesis of Poly-Lysine Oleate Based on Basodrill™ S 100

An aqueous solution of poly-lysine (9.934 kg) having a K value of 11 (Mw=17.100 g/mol, trade name Basodrill™ S100 by BASF) was dosed into the reactor. Successively water was removed from the solution at 160° C. Then oleic acid (Edenor™ T105, 0.71 kg) was added to the reaction mixture and water was removed from the reaction mixture at 160° C. for 240 min. The reaction had to be stopped due to too high viscosity of the melt.

### Example 8: Storage Stability of Solid-Based Formulation

Particle size distributions in example 8-11 were determined by CIPAC method MT 46-accelerated storage procedure.

The following concentrated solid-based composition was prepared:

Component D: 25% w/w azoxystrobin

Component A+B: 2.5% w/w poly-lysine oleate (5% oleic acid) in water—calculated to 100% active

Liquid comprising salt was added, wherein the liquid comprised Castoroil ethoxylate+Calciumdodecyl-benzenesulfonate (Agnique CSO 30+Agnique ABS 70 C) 2.5% in the concentrated solid-based composition, calculated to 100% active, wherein Agnique CSO 30: Agnique ABS 70 C was 3:1.

Then add water up to 100%. The concentrated solid-based composition was milled by wet comminution and evaluated.

Note: if sheer-sensitive salts are used, milling may take place before addition of said salts.

Particle Size Stability of the solid-based composition:

| | | μm |
|---|---|---|
| start | dx10 | 0.72 |
| | dx50 | 1.55 |
| | dx90 | 3.41 |
| 14 days/RT | dx10 | 0.77 |
| | dx50 | 1.85 |
| | dx90 | 3.94 |
| 14 days/54° C. | dx10 | 0.81 |
| | dx50 | 2.05 |
| | dx90 | 4.89 |

The concentrated solid-based composition was diluted to give a spray-mix: 5% w/w concentrated solid-based composition+95% w/w CIPAC D water (hard water).

Suspensibility was determined by CIPAC method MT 161.

Suspensibility test in the spray solution CIPAC D water:

| start | |
|---|---|
| Blooming | homogenous |
| suspensibility after 30 min | 92.29% |
| 14 days/RT | |
| blooming | homogenous |
| suspensibility after 30 min | 93.73% |
| 14 days/54° C. | |
| blooming | homogenous |
| suspensibility after 30 min | 84.1% |

### Example 9: Storage Stability of Solid-Based Formulation

The following concentrated solid-based composition was prepared:

Component D: 25% w/w azoxystrobin

Component A+B: 2.5% w/w poly-lysine oleate (5% oleic acid) in water—calculated to 100% active

A solution comprising salt was added, wherein the solution comprised 62% Glyphosate IPA-Salt in water. The concentrated solid-based composition comprised 40% Glyphosate IPA-salt (calculated to 100% active).

The solid-based composition was milled by wet comminution and evaluated.

Particle Size Stability of the concentrated solid-based composition

| | | |
|---|---|---|
| start | dx10 | 0.67 |
| | dx50 | 1.47 |
| | dx90 | 3.17 |
| 14 days/RT | dx10 | 0.697 |
| | dx50 | 1.500 |
| | dx90 | 3.180 |
| 14 days/54° C. | dx10 | 0.71 |
| | dx50 | 1.54 |
| | dx90 | 3.25 |

The concentrated solid-based composition was diluted to give a spray-mix: 5% w/w concentrated solid-based composition+95% w/w CIPAC D water (hard water).

Suspensibility was determined by CIPAC method MT 161.

Suspensibility test in the spray mix:

| start | |
|---|---|
| blooming | homogenous |
| Suspensibility after 30 min | 89.18% |
| 14 days/RT | |
| blooming | homogenous |
| Suspensibility after 30 min | 88.32% |
| 14 days/54° C. | |
| blooming | homogenous |
| Suspensibility after 30 min | 88.67% |

### Example 10: Storage Stability of Solid-Based Formulation

The following concentrated solid-based composition (SC) was prepared:

Component D: 20% w/w azoxystrobin

Component A+B: 2.5% w/w poly-lysine oleate (5% oleic acid) in water—calculated to 100% active

Component C: 0.86% w/w defoamer

Add water up to 100% w/w; the SC was milled by wet comminution.

An emulsion comprising salt was prepared, wherein the emulsion comprised 25% w/w Oxyfluorofen, 53% w/w Agnique AMD 10 (solvent), 10% w/w Solvesso 200 ND (Co-Solvent), 10% w/w Agnique CSO 35 and 2% Agnique ABS 70C.

The SC and the emulsion were mixed in various ratios and the particle size of component D was determined.

Mixture matrix and particle size stability at room temperature (the particle size was determined before storage and after storage for 2 weeks at room temperature):

| | Particle Size dx50 |
|---|---|
| Ratios | constant: Initial vs. |
| SC:emulsion | RT after 2 w [μm] |
| 1:1 | 1.67 |
| 2:1 | 1.61 |
| 3:1 | 1.74 |
| 4:1 | 1.77 |
| 5:1 | 1.9 |
| 6:1 | 2.05 |
| 7:1 | 2.23 |

The compositions comprising various ration SC: emulsion were diluted to give a spray-mix: 5% w/w composition+95% w/w of either CIPAC D water (hard water) or CIPAC B water (soft water).

The spray-mixes comprising either CIPAC D or B were evaluated according to CIPAC method MT 161.

CIPAC B (Soft Water):

| Ratios | |
|---|---|
| SC:emulsion | Residue [g] |
| 1:1 | 0.13 |
| 2:1 | 0.13 |
| 3:1 | 0.13 |
| 4:1 | 0.34 |
| 5:1 | 0.76 |
| 6:1 | 0.76 |
| 7:1 | 0.67 |

CIPAC D (Hard Water):

| Ratios | |
|---|---|
| SC:emulsion | Residue [g] |
| 1:1 | 0.66 |
| 2:1 | 0.56 |
| 3:1 | 0.13 |
| 4:1 | 0.12 |
| 5:1 | 0.11 |
| 6:1 | 0.1 |
| 7:1 | 0.11 |

A small residue [g] value in comparison to the amount of component D present in the spray-mix indicates a homogenous distribution of component D within the spray-mix

### Example 11: Storage Stability of Solid-Based Formulation

The following concentrated solid-based composition was prepared:

Component D: 25% w/w azoxystrobin

Component A+B: 2.5% w/w poly-lysine oleate (5% oleic acid) in water—calculated to 100% active

A solution 1 comprising salt was added, wherein the solution 1 comprised 62% Glyphosate IPAS-alt in water. The concentrated solid-based composition comprised 40% Glyphosate IPA-salt (calculated to 100% active).

The solid-based composition was milled by wet comminution and evaluated.

A solution 2 comprising salt was prepared, wherein the solution comprised Fertilzer NPK 10-34-0 and 40% w/w water (product used: Ammonium polyphosphate solution from BASF North America).

The concentrated solid-based composition was diluted to give a spray-mix: 5% w/w concentrated solid-based composition+10-95% solution 2+0-85% w/w of either CIPAC D water (hard water) or CIPAC B water (soft water).

Formulation 1:

85% CIPAC water B or D

10% solution 2

5% concentrated solid-based composition

CIPAC water and solution 2 were mixed before the concentrated solid-based composition was added.

Evaluation of the formulation was done according to CIPAC method MT 161: Suspension was not stable within 30 minutes at room temperature, due to settling of component D.

Formulation 2:

0-75% CIPAC water B or D

20-95% solution 2

5% concentrated solid-based composition

CIPAC water and solution 2 were mixed before the concentrated solid-based composition was added.

Evaluation of the formulation was done according to CIPAC method MT 161: Suspension kept homogenous within 30 minutes at room temperature—no settling occurred.

1. A polymeric stabilizing agent selected from poly-lysine derivatives obtained by a process comprising the steps of:
I. heating an aqueous lysine solution to boiling;
II. increasing a temperature of the aqueous lysine solution to a reaction temperature in the range of about 105° C. to about 180° C.;
III. keeping the reaction temperature in the range of about 105° C. to about 180° C. until;
i. a melt viscosity of the reaction mixture is in the range of about 350 mPa*s to about 6,500 mPa*s when measured at 160° C. and
ii. an amine number in the range of about 100 mg KOH/g to about 500 mg KOH/g is achieved;
IV. optionally, releasing a vacuum applied;
V. adding alkyl-carboxylic acid or alkenyl-carboxylic acid in amounts of 2.5 mol % to 10 mol %, relative to a theoretical amount of poly-lysine in the reaction mixture; and
VI. increasing or keeping the reaction temperature in the range of about 105° C. to about 180° C. until number of free alkyl-carboxylic acid or alkenyl-carboxylic acid is 9% by weight, relative to the total weight of the reaction mixture,
wherein vacuum is applied in step (a), (b), and/or (c), and water is removed continuously during the process.

2. A storage-stable solid-based composition comprising:
I. a liquid phase comprising components A and B, one or more salts, optionally component C, and optionally at least one additional solvent, wherein component B comprises at least one solvent in which component A is soluble, wherein component C is selected from at least one additional compound, wherein the at least one additional solvent is immiscible with component B, and wherein component A and at least one salt of the one or more salts are soluble in component B, and
II. component D comprising at least one solid compound, wherein component D is dispersed in the liquid phase,
wherein component A comprises at least one poly-lysine derivative obtained by a process comprising the steps of:
(a) heating an aqueous lysine solution to boiling,
(b) increasing a temperature of the aqueous lysine solution to a reaction temperature in the range of about 105° C. to about 180° C.,
(c) keeping the reaction temperature in the range of about 105° C. to about 180° C. until:
i. a melt viscosity of the reaction mixture is in the range of about 350 mPa*s to about 6,500 mPa*s when measured at 160° C., and
ii. an amine number in the range of about 100 mg KOH/g to about 500 mg KOH/g is achieved,
(d) optionally, releasing a vacuum applied,
(e) adding alkyl-carboxylic acid or alkenyl-carboxylic acid in amounts of 2.5 mol % to 10 mol %, relative to a theoretical amount of poly-lysine in the reaction mixture
(f) increasing or keeping the reaction temperature in the range of about 105° C. to about 180° C. until number of free alkyl-carboxylic acid or alkenyl-carboxylic acid is 9% by weight, relative to the total weight of the reaction mixture,
wherein vacuum is applied in step (a), (b) and/or (c) and water is removed continuously during the process.

3. The storage-stable solid-based composition of claim 2, wherein the one or more salts in the liquid phase are soluble in component B or a solvent miscible with component B at 20° C. and 101.3 kPa until the saturation concentration is achieved.

4. The storage-stable solid-based composition of claim 2, wherein the one or more salts in the liquid phase (1) are soluble in the at least one additional solvent at 20° C. and 101.3 kPa until the saturation concentration is achieved.

5. The storage-stable solid-based composition of claim 2, wherein at least one salt of the one or more salts dissociates in the liquid phase into ions, wherein both the cation and the anion are hydrophilic.

6. The storage-stable solid-based composition of claim 2, wherein at least one salt of the one or more salts in the liquid phase dissociates in the liquid phase into ions, wherein the cation or the anion is amphiphilic.

7. The storage-stable solid-based composition of claim 2, wherein the liquid phase and/or component D comprises at least one agrochemically active compound.

8. The storage-stable solid-based composition of claim 2, wherein at least one of the solid compounds in component D is selected from agrochemically active compounds insoluble in component B.

9. The storage-stable solid-based composition of claim 2, wherein the composition is stable at 20° C. and/or 54° C. for 14 days.

10. A method for production of a solid-based composition according to claim 2, the method comprising mixing in no specified order in one or more steps components A, B, optionally C, component D, and one or more salts which are soluble in component B or a solvent miscible with component B.

11. The method of claim 11, wherein the pH is adjusted of both the composition and/or solution comprising component A and the composition and/or solution comprising at least one salt soluble in component B or a solvent miscible with component B, before the composition and/or solution comprising component A and the composition and/or solution comprising at least one salt soluble in component B or a solvent miscible with component B are mixed with each other.

12. The method of claim 10 further comprising the steps of:
I. providing a solution (1) of poly-lysine functionalized with fatty acid(s) by mixing at least components A and B, and optionally adjusting to pH of the solution (1), and
II. providing a liquid (2) by mixing in no specified order in one or more steps one or more salts in at least one solvent, and optionally adjusting the pH of the liquid (2), and
III. providing a solid-based composition (3) by dispersing component D in a dispersing medium comprising at least one dispersant in which component D is insoluble, and
IV. mixing at least the solution (1) and the solid-based composition (3) and optionally the liquid (2).

13. A method of producing a tank-mix, the method comprising diluting the storage-stable composition of claim 2 with soft and/or hard water, wherein the water optionally comprises fertilizer.

14. A method of stabilizing a solid-based composition, the method comprising the steps of adding to the solid-based composition at least one poly-lysine derivative obtained by a process comprising the steps of:
(a) heating an aqueous lysine solution to boiling;
(b) increasing a temperature of the aqueous lysine solution to a reaction temperature in the range of about 105° C. to about 180° C.;
(c) keeping the reaction temperature in the range of about 105° C. to about 180° C. until:
i. a melt viscosity of the reaction mixture is in the range of about 350 mPa*s to about 6,500 mPa*s when measured at 160° C.; and
ii. an amine number in the range of about 100 mg KOH/g to about 500 mg KOH/g is achieved;
(d) optionally, releasing a vacuum applied;
(e) adding alkyl-carboxylic acid or alkenyl-carboxylic acid in amounts of 2.5 mol % to 10 mol %, relative to a theoretical amount of poly-lysine in the reaction mixture; and
(f) increasing or keeping the reaction temperature in the range of about 105° C. to about 180° C. until number of free alkyl-carboxylic acid or alkenyl-carboxylic acid is ≤9% by weight, relative to the total weight of the reaction mixture,
wherein vacuum is applied in step (a), (b), and/or (c), and water is removed continuously during the process.

15. A method to increase storage-stability of a solid-based composition comprising one or more salts, the method comprising the step of adding at least one poly-lysine derivative to the solid-based composition, wherein at least one poly-lysine derivative is obtained by a process comprising the steps of:
(a) heating an aqueous lysine solution to boiling,
(b) increasing a temperature of the aqueous lysine solution to a reaction temperature in the range of about 105° C. to about 180° C.,
(c) keeping the reaction temperature in the range of about 105° C. to about 180° C. until:
i. a melt viscosity of the reaction mixture is in the range of about 350 mPa*s to about 6,500 mPa*s when measured at 160° C., and
ii. an amine number in the range of about 100 mg KOH/g to about 500 mg KOH/g is achieved;
(d) optionally, releasing a vacuum applied;
(e) adding alkyl-carboxylic acid or alkenyl-carboxylic acid in amounts of 2.5 mol % to 10 mol %, relative to a theoretical amount of poly-lysine in the reaction mixture;
(f) increasing or adding the reaction temperature in the range of about 105° C. to about 180° C. until number of free alkyl-carboxylic acid or alkenyl-carboxylic acid is ≤9% by weight, relative to the total weight of the reaction mixture,
wherein vacuum is applied in step (a), (b), and/or (c), and water is removed continuously during the process, and wherein the solid-based composition comprises at least one solvent miscible with the one or more salts.
